(19) United States
(12) Patent Application Publication
(10) Pub. No.: US 2026/0356805 A1
(43) Pub. Date: Nov. 12, 2026

(54) Process for the Preparation of Tert-Butyl (2-Azabicyclo[2.2.1]Heptan-4-yl)Carbamate and Related Compounds

(71) Applicant: Bristol-Myers Squibb Company, Princeton, NJ (US)

(72) Inventors: Jianqing Li, Southborough, MA (US); Arvind Mathur, Bridgewater, NJ (US); Ning Li, Lexington, MA (US); Oleg Epstein, Belmont, MA (US); Rulin Zhao, Pennington, NJ (US); Bei Wang, Newtown, PA (US); Zhenqiu Hong, Princeton, NJ (US)

(73) Assignee: Bristol-Myers Squibb Company, Princeton, NJ (US)

(21) Appl. No.: 19/111,532

(22) PCT Filed: Sep. 20, 2023
(86) PCT No.: PCT/US2023/074645

§ 371 Date: Mar. 13, 2025

Related U.S. Application Data
(60) Provisional application No. 63/408,695, filed on Sep. 21, 2022.

(51) Int. Cl. C07D 221/22

(52) U.S. Cl. CPC C07D 221/22

(57) ABSTRACT

Provided herein are methods for preparing 2-azabicyclo[2.2.1]heptan-4-amine, a key structural motif found in a number of synthetic compounds that are inhibitors of rho-associated protein kinase, and its primary amine-protected version tert-butyl (2-azabicyclo[2.2.1]heptan-4-yl)carbamate, and enantiomers thereof. Also provided herein are novel intermediate compounds and their enantiomers for use in preparing the aforementioned target 2-azabicyclo[2.2.1]heptanyl compounds.

### CROSS REFERENCE TO RELATED APPLICATIONS

This application claims the benefit of priority of U.S. Provisional Application No. 63/408,695, filed Sep. 21, 2022, which is incorporated by reference herein in its entirety for any purpose.

### FIELD

The present disclosure relates generally to methods of preparing tert-butyl (2-azabicyclo[2.2.1]heptan-4-yl)carbamate, its enantiomers, and novel intermediate compounds.

### BACKGROUND

The compound 2-azabicyclo[2.2.1]heptan-4-amine (designated herein as a compound of Formula T-1) is a key structural motif found in a number of synthetic compounds that are inhibitors of rho-associated protein kinase. These inhibitors are potentially useful for treating or preventing various diseases or conditions. See, e.g., US Publication No. 2011/0144150. The chemical structure of a compound of Formula T-1 is shown below:

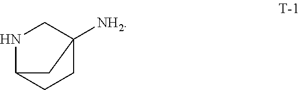

Selectively protected versions of a compound of Formula T-1 and its enantiomers are of interest as building blocks for various compounds. Accordingly, in one aspect, provided herein are methods for preparing tert-butyl (2-azabicyclo[2.2.1]heptan-4-yl)carbamate, its enantiomers, and salts thereof. The chemical structure of a compound of Formula T-2, which is a primary amine-protected version of a compound of Formula T-1, is shown below:

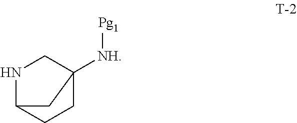

Also provided herein are intermediate compounds for use in preparing T-1 and/or T-2.

### SUMMARY

Described herein, in certain embodiments, are methods of preparing tert-butyl (2-azabicyclo[2.2.1]heptan-4-yl)carbamate, its enantiomers, and salts thereof as building blocks for various inhibitor compounds. Also described herein are intermediate compounds for use in preparing said compounds.

The present embodiments can be understood more fully by reference to the detailed description and examples, which are intended to exemplify non-limiting embodiments.

In one aspect, provided herein are methods of preparing a compound of Formula T-2 or a salt thereof.

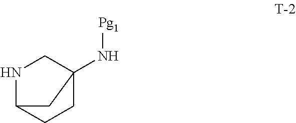

In another aspect, provided herein are methods of preparing compounds of Formula T-3 and T-4, which are enantiomers of a compound of Formula T-2, or salts thereof.

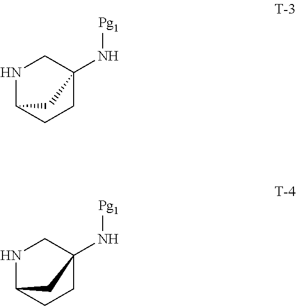

In certain aspects, provided herein are methods of preparing a compound of Formula T-2:

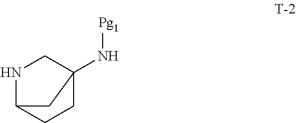

or a salt thereof, comprising the following steps:
(a) reacting a compound of Formula 1:

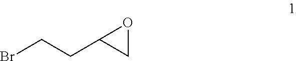

or a salt thereof, with a compound of Formula 2:

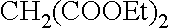

or a salt thereof, to form a compound of Formula 3:

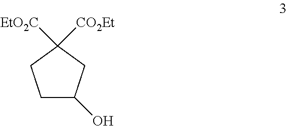 [SMILES: CCOC(=O)C1(C(=O)OCC)CCC(O)C1]

or a salt thereof;
(b) reacting a compound of Formula 3, or a salt thereof, with phthalimide to form a compound of Formula 4:

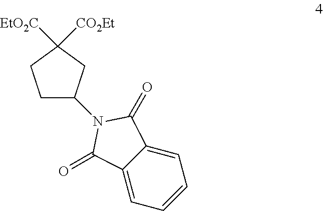 [SMILES: CCOC(=O)C1(C(=O)OCC)CCC(N2C(=O)c3ccccc3C2=O)C1]

or a salt thereof;
(c) deprotecting a compound of Formula 4, or a salt thereof, to form a compound of Formula 5:

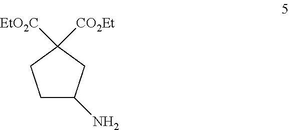 [SMILES: CCOC(=O)C1(C(=O)OCC)CCC(N)C1]

or a salt thereof;
(d) reacting a compound of Formula 5, or a salt thereof, with a base to form a compound of Formula 6:

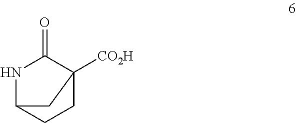 [SMILES: O=C(O)C12CCC(C1)NC2=O]

or a salt thereof;
(e) reacting a compound of Formula 6, or a salt thereof, with R₃OH, in the presence of diphenylphosphoryl azide and an amine base acid to form a compound of Formula 7:

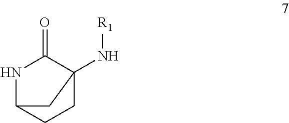

or a salt thereof; and
(f) reacting a compound of Formula 7, or a salt thereof, with Pg₂X to form a compound of Formula 8:

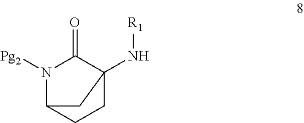

or a salt thereof; and
(g) reducing a compound of Formula 8, or a salt thereof, to form a compound of Formula 9:

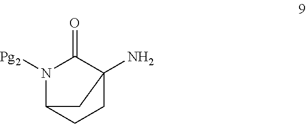

or a salt thereof; and
(h) reducing a compound of Formula 9, or a salt thereof, to form a compound of Formula 10:

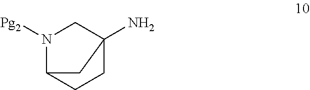

or a salt thereof; and
(i) protecting the primary amine group of a compound of Formula 10, or a salt thereof, to form a compound of Formula 11:

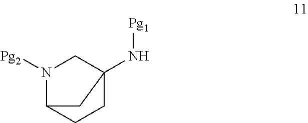

or a salt thereof; and
(j) reducing a compound of Formula 11, or a salt thereof, to form a compound of Formula T-2.

In certain aspects, also provided herein are methods of preparing a compound of Formula T-2:

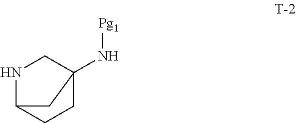

or a salt thereof, comprising the following steps:
(a) deprotecting and subsequently protecting the primary amine group of a compound of Formula 7:

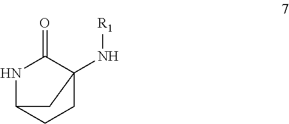

or a salt thereof, to form a compound of Formula 7.5:

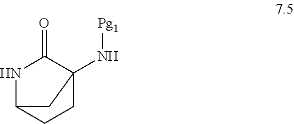

(b) hydrolyzing a compound of Formula 7.5, or a salt thereof, to form a compound of Formula 8.5:

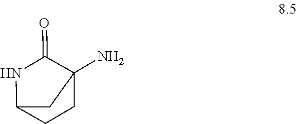

or a salt thereof; and
(c) reducing a compound of Formula 8.5, or a salt thereof, to form a compound of Formula 9.5:

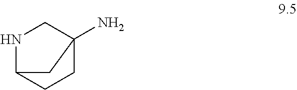

or a salt thereof; and
(d) sequentially protecting the substituent and heterocyclic amines of a compound of Formula 9.5, or a salt thereof, and selectively deprotecting the substituent amine to form a compound of Formula 10:

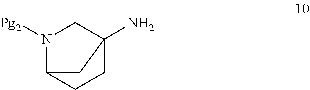

or a salt thereof.

### DETAILED DESCRIPTION

### Definitions

Unless defined otherwise, all technical and scientific terms used herein have the same meaning as is commonly understood by one of skill in the art to which this disclosure belongs. In the following description, certain specific details are set forth in order to provide a thorough understanding of various embodiments of the disclosure. It is to be understood that the foregoing general description and the following detailed description are exemplary and explanatory only and are not restrictive of any subject matter claimed. To the extent any material incorporated herein by reference is inconsistent with the express content of this disclosure, the express content controls. In this application, the use of the singular includes the plural unless specifically stated otherwise. It must be noted that, as used in the specification and the appended claims, the singular forms “a,” “an”, and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Unless the context requires otherwise, throughout the present specification and claims, the word “comprise” and variations thereof, such as, “comprises” and “comprising” are to be construed in an open, inclusive sense, that is, as “including, but not limited to”.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean±20%, ±10%, ±5%, or ±1% of the indicated range, value, or structure, unless otherwise indicated.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, the term “salt” refers to acid or base salts of the compounds disclosed herein. It is understood that “pharmaceutically acceptable salts” are non-toxic. Non-limiting examples of pharmaceutically acceptable salts include acid addition salts and base addition salts.

Pharmaceutically acceptable acid addition salts are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

Pharmaceutically acceptable base addition salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Non-limiting examples of inorganic salts include ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, non-limiting examples of which include ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins, and the like.

“Optional” or “optionally” means that the subsequently described event of circumstances may or may not occur, and that the description includes instances where said event or circumstance occurs and instances in which it does not. For example, “optionally converting Compound X to a salt thereof” means that Compound X may or may not be converted to a salt. In some embodiments, Compound X is converted to a salt, whereas in other embodiments Compound X is not converted to a salt.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Synthesis Route 1

In one aspect, provided herein is a method of preparing a compound of Formula T-2 or a salt thereof accordingly to the synthetic route outlined in Scheme 1 (“Synthesis Route 1”).

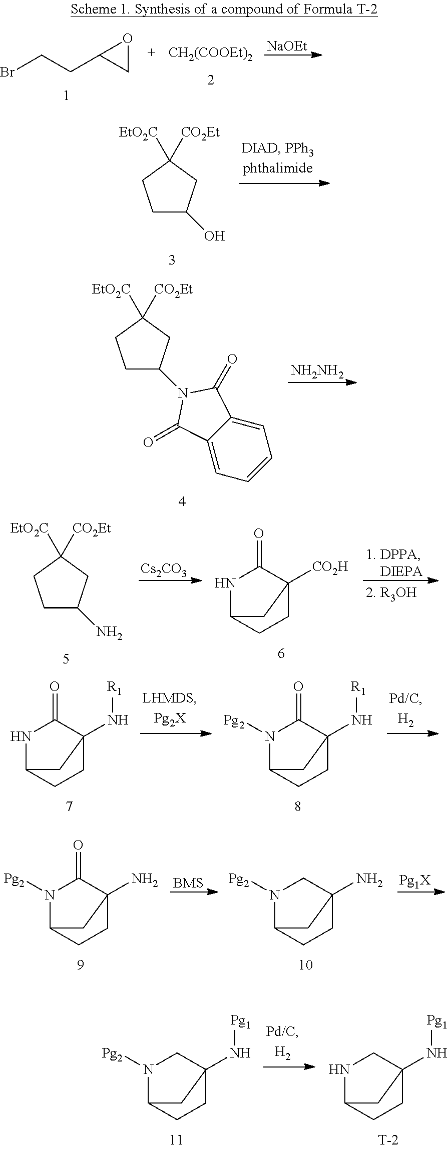

Thus, in certain embodiments, disclosed herein are methods of preparing a compound of Formula T-2:

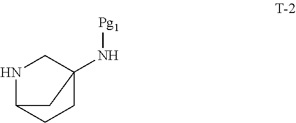

or a salt thereof, comprising the following steps:
(a) reacting a compound of Formula 1:

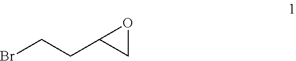

or a salt thereof, with a compound of Formula 2:

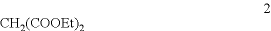

or a salt thereof, to form a compound of Formula 3:

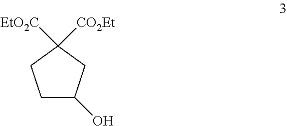 [SMILES: CCOC(=O)C1(C(=O)OCC)CCC(O)C1]

or a salt thereof;
(b) reacting a compound of Formula 3, or a salt thereof, with phthalimide to form a compound of Formula 4:

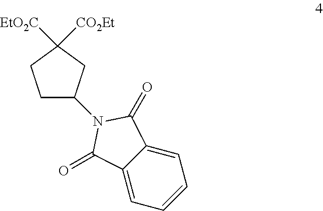 [SMILES: CCOC(=O)C1(C(=O)OCC)CCC(N2C(=O)c3ccccc3C2=O)C1]

or a salt thereof;
(c) deprotecting a compound of Formula 4, or a salt thereof, to form a compound of Formula 5:

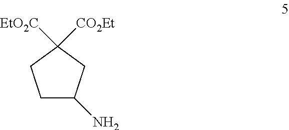 [SMILES: CCOC(=O)C1(C(=O)OCC)CCC(N)C1]

or a salt thereof;
(d) reacting a compound of Formula 5, or a salt thereof, with a base to form a compound of Formula 6:

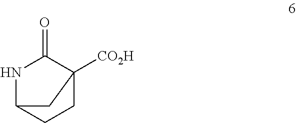 [SMILES: O=C(O)C12CCC(C1)NC2=O]

or a salt thereof;
(e) reacting a compound of Formula 6, or a salt thereof, with R₃OH to form a compound of Formula 7:

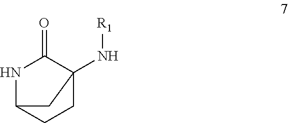

or a salt thereof; and
(f) reacting a compound of Formula 7, or a salt thereof, with Pg₂X to form a compound of Formula 8:

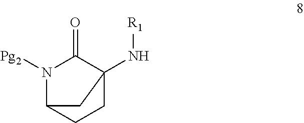

or a salt thereof; and
(g) reducing a compound of Formula 8, or a salt thereof, to form a compound of Formula 9:

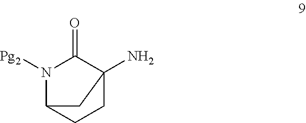

or a salt thereof; and
(h) reducing a compound of Formula 9, or a salt thereof, to form a compound of Formula 10:

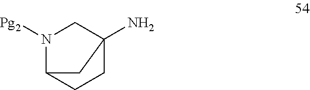

or a salt thereof; and
(i) protecting the primary amine group of a compound of Formula 10, or a salt thereof, to form a compound of Formula 11:

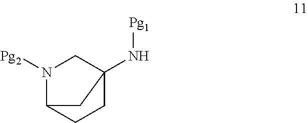

or a salt thereof; and
(j) reducing a compound of Formula 11, or a salt thereof, to form a compound of Formula T-2.

In certain embodiments, step (a) is carried out in the presence of a base. In some embodiments, the base is sodium hydride, potassium tert-butoxide, or sodium ethoxide. In other embodiments, the base is sodium ethoxide.

In certain embodiments, step (a) is carried out using an alcohol as solvent. In some embodiments, the alcohol is methanol, ethanol, or isopropanol. In certain embodiments, the alcohol is ethanol.

In certain embodiments, step (a) is carried out at a temperature of between about 0-30 °C. In some embodiments, step (a) is carried out at a starting temperature of about 0-10 °C, then at 20-30 °C.

In certain embodiments, step (a) is carried out with the compound of Formula 1, or a salt thereof, that is separated into enantiomers, Formula 1a and Formula 1b, or salts thereof, using kinetic resolution. In certain embodiments, the kinetic resolution is Jacobsen's kinetic resolution. In certain embodiments, the kinetic resolution is hydrolytic kinetic resolution catalyzed by chiral cobalt-salen complexes.

In certain embodiments, step (b) further comprises triphenylphosphine and diisopropyl azodicarboxylate.

In certain embodiments, step (b) is carried out using an aprotic solvent. In some embodiments, the aprotic solvent is diethyl ether, tetrahydrofuran, 2-methyltetrahydrofuran, acetonitrile, 1,4-dioxane, toluene, dichloromethane, dichloroethane, chloroform, or a mixture thereof. In certain embodiments, the aprotic solvent is tetrahydrofuran.

In certain embodiments, step (b) is carried out at a temperature of between about 0-30 °C. In some embodiments, step (b) is carried out at a starting temperature of about 0-10 °C, then at 20-30 °C.

In certain embodiments, step (c) is carried out in the presence of a base. In some embodiments, the base is sodium borohydride, methylamine, methylhydrazine, or hydrazine. In other embodiments, the base is hydrazine.

In certain embodiments, step (c) is carried out using a mixture of alcohol and an organic solvent. In some embodiments, the alcohol is methanol, ethanol, or isopropanol. In some embodiments, the organic solvent is dimethyl sulfoxide, dichloromethane, tetrahydrofuran, or 2-methyltetrahydrofuran. In certain embodiments, the alcohol is methanol and the organic solvent is dichloromethane.

In certain embodiments, step (d) is carried out using an inorganic base. In some embodiments, the inorganic base is sodium tert-butoxide, potassium tert-butoxide, potassium carbonate, or cesium carbonate. In other embodiments, the inorganic base is cesium carbonate.

In certain embodiments, step (d) is carried out using an alcohol as solvent. In some embodiments, the alcohol is methanol, ethanol, or isopropanol. In certain embodiments, the alcohol is methanol.

In certain embodiments, step (d) is carried out at a temperature of between about 25-80 °C. In certain embodiments, step (d) is carried out at a temperature of between about 50-70 °C.

In certain embodiments, step (e) is carried out using an amine base. In some embodiments, the amine base is 2,2,6,6-tetramethylpiperidine, trimethylamine, triethylamine, or N,N-diisoproypylethylamine. In other embodiments, the amine base is N,N-diisoproypylethylamine.

In certain embodiments, step (e) is carried out using an organic solvent. In some embodiments, the organic solvent is dimethyl sulfoxide, dichloromethane, tetrahydrofuran, 2-methyltetrahydrofuran, or a mixture thereof. In certain embodiments, the organic solvent is 2-methyltetrahydrofuran.

In certain embodiments, step (e) is carried out at a starting temperature of between about 40-70 °C, then at a reflux temperature above 50-120 °C. In certain embodiments, step (e) is carried out at a starting temperature of about 50-60 °C, then at a reflux temperature above 70-100 °C.

In certain embodiments, step (e) is carried out at a temperature of between about 40-70 °C, then at a temperature between about 50-120 °C. In certain embodiments, step (e) is carried out at a temperature of about 50-60 °C or about 50-70 °C, then at a temperature between about 70-100 °C.

In certain embodiments, following step (e), the compound of Formula 7, or a salt thereof, is separated into enantiomers, Formula 7a and Formula 7b, or salts thereof, using super fluid chromatography.

In certain embodiments, step (f) is carried out in the presence of a non-nucleophilic base. In some embodiments, the non-nucleophilic base is N,N-diisopropylethylamine, lithium diisopropylamide, sodium bis(trimethylsilyl)amide, sodium tert-butoxide, or lithium bis(trimethylsilyl)amide. In other embodiments, the non-nucleophilic base is lithium bis(trimethylsilyl)amide.

In certain embodiments, step (f) is carried out using an ether solvent. In some embodiments, the ether solvent is tetrahydrofuran, 2-methyltetrahydrofuran, 1,4-dioxane, methyl tert-butyl ether, or a mixture thereof. In certain embodiments, the ether solvent is tetrahydrofuran.

In certain embodiments, step (g) is catalyzed by a transition metal. In some embodiments, the transition metal is palladium chloride, palladium on barium sulfate, or palladium on carbon (Pd/C). In other embodiments, the transition metal is palladium on carbon (Pd/C).

In certain embodiments, step (g) is carried out using an alcohol as solvent. In some embodiments, the alcohol is methanol, ethanol, or isopropanol. In certain embodiments, the alcohol is methanol.

In certain embodiments, step (h) is carried out using a reducing agent. In some embodiments, the reducing agent is a metal hydride, aluminum hydride, borohydride, or borane reagent. In some embodiments, the reducing agent is lithium aluminum hydride, triphenylphosphine borane, borane tetrahydrofuran, or borane dimethylsulfide. In other embodiments, the reducing agent is borane dimethylsulfide.

In certain embodiments, step (h) is carried out using an aprotic solvent. In some embodiments, the aprotic solvent is diethyl ether, tetrahydrofuran, 2-methyltetrahydrofuran, acetonitrile, 1,4-dioxane, toluene, dichloromethane, dichloroethane, chloroform, or a mixture thereof. In certain embodiments, the aprotic solvent is tetrahydrofuran.

In certain embodiments, step (h) is carried out at a temperature of between about 25-80 °C. In certain embodiments, step (h) is carried out at a temperature of about 60-70 °C.

In certain embodiments, step (i) is carried out using an organic solvent. In some embodiments, the organic solvent is chloroform, dichloroethane, toluene, methanol, ethanol, isopropanol, or 2-methyl-THF, dioxane, ethyl acetate, or tetrahydrofuran. In certain embodiments, the organic solvent is dichloromethane, methanol, toluene, or tetrahydrofuran.

In certain embodiments, step (j) is catalyzed by a transition metal. In some embodiments, the transition metal is palladium chloride, palladium on barium sulfate, or palladium on carbon (Pd/C). In other embodiments, the transition metal is palladium on carbon (Pd/C).

In certain embodiments, step (j) is carried out using an alcohol as solvent. In some embodiments, the alcohol is methanol, ethanol, or isopropanol. In certain embodiments, the alcohol is methanol.

### Synthesis Route 2

In another aspect, provided herein is a method of preparing the compound Formula T-2 or a salt thereof accordingly to the synthetic route outlined in Scheme 2 (“Synthesis Route 2”).

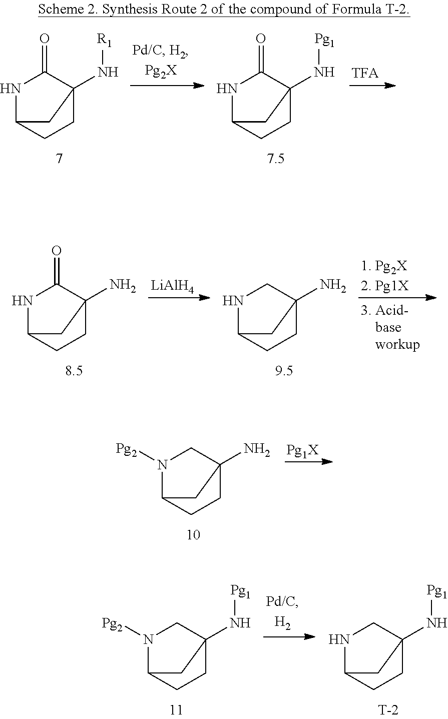

Thus, in certain embodiments, disclosed herein are methods of preparing a compound of Formula T-2:

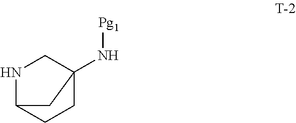

or a salt thereof, comprising the following steps:
(a) deprotecting and subsequently protecting the primary amine group of a compound of Formula 7:

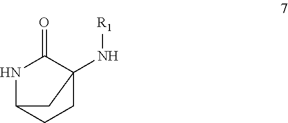

or a salt thereof, to form a compound of Formula 7.5:

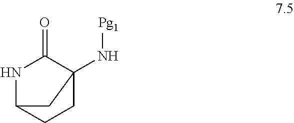

(b) hydrolyzing a compound of Formula 7.5, or a salt thereof, to form a compound of Formula 8.5:

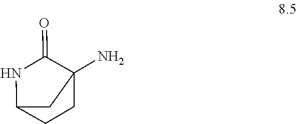

or a salt thereof; and
(c) reducing a compound of Formula 8.5, or a salt thereof, to form a compound of Formula 9.5:

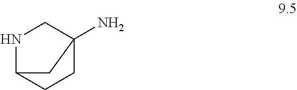

or a salt thereof; and
(d) sequentially protecting the substituent and heterocyclic amines of a compound of Formula 9.5, or a salt thereof, and selectively deprotecting the substituent amine to form a compound of Formula 10:

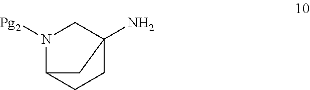

or a salt thereof.

In certain embodiments, step (a) is catalyzed by a transition metal. In some embodiments, the transition metal is palladium chloride, palladium on barium sulfate, or palladium on carbon (Pd/C). In other embodiments, the transition metal is palladium on carbon (Pd/C).

In certain embodiments, step (a) is carried out using an alcohol as solvent. In some embodiments, the alcohol is methanol, ethanol, or isopropanol. In certain embodiments, the alcohol is methanol.

In certain embodiments, step (a) is carried out using an organic solvent. In some embodiments, the organic solvent is chloroform, dichloromethane, tetrachloromethane, dichloroethane, toluene, methanol, ethanol, isopropanol, toluene, or tetrahydrofuran. In certain embodiments, the organic solvent is dichloromethane, methanol, toluene, or tetrahydrofuran.

In certain embodiments, step (b) is carried out with an acid. In some embodiments, the acid is trifluoroacetic acid, hydrochloride, or phosphoric acid. In certain embodiments, the acid is trifluoroacetic acid.

In certain embodiments, step (b) is carried out using an organic solvent. In some embodiments, the organic solvent is chloroform, dichloromethane, tetrachloromethane, dichloroethane, toluene, methanol, ethanol, isopropanol, toluene, or tetrahydrofuran. In certain embodiments, the organic solvent is dichloromethane, methanol, toluene, or tetrahydrofuran.

In certain embodiments, step (b) is carried out at a temperature of about 20-30 °C.

In certain embodiments, step (c) is carried out using a reducing agent. In some embodiments, the reducing agent is a metal hydride, aluminum hydride, borohydride, or borane reagent. In some embodiments, the reducing agent is lithium aluminum hydride, triphenylphosphine borane, borane tetrahydrofuran, or borane dimethylsulfide. In other embodiments, the reducing agent is lithium aluminum hydride.

In certain embodiments, step (c) is carried out using an aprotic solvent. In some embodiments, the aprotic solvent is diethyl ether, tetrahydrofuran, 2-methyltetrahydrofuran, acetonitrile, 1,4-dioxane, toluene, dichloromethane, dichloroethane, chloroform, or a mixture thereof. In certain embodiments, the aprotic solvent is diethyl ether, tetrahydrofuran, or a mixture thereof.

In certain embodiments, step (d) is carried out using an organic solvent. In some embodiments, the organic solvent is chloroform, dichloromethane, tetrachloromethane, dichloroethane, toluene, methanol, ethanol, isopropanol, toluene, or tetrahydrofuran. In certain embodiments, the organic solvent is dichloromethane, methanol, toluene, or tetrahydrofuran.

In certain embodiments, the selective deprotection of the substituent amine in step (d) is carried out using an acid. In some embodiments, the acid is hydrochloric acid.

### Compounds

In one aspect, provided herein is a compound of Formula T-2:

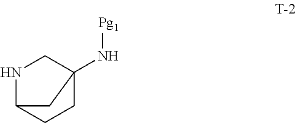

or a salt thereof, wherein Pg₁ is a first protecting group.

In certain embodiments, Pg₁ is tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, or fluorenylmethyloxycarbonyl. In certain embodiments, Pg₁ is tert-butyloxycarbonyl.

In certain embodiments, the compound of Formula T-2 is separated into enantiomers, compounds of Formula T-3 and Formula T-4:

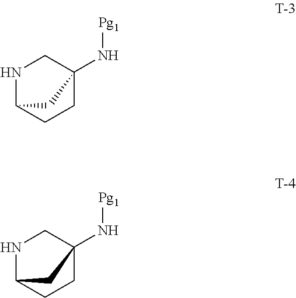

or salts thereof.

In one aspect, provided herein are novel intermediate compounds that are useful in the synthesis of the compound of Formula T-2 or a salt thereof. In certain embodiments, the novel intermediate compound is:

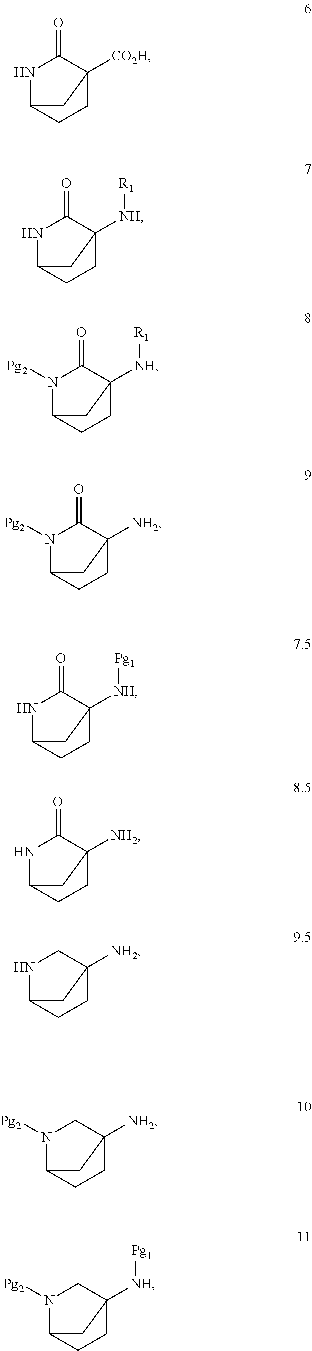

or a salt thereof.

In certain embodiments, Pg₂ is a second protecting group. In certain embodiments, Pg₂ is tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, or fluorenylmethyloxycarbonyl. In certain embodiments, Pg₂ is a benzyl group optionally substituted by C₁-C₆ alkoxy, C₁-C₆ alkyl, or halide.

In certain embodiments, R₁ is a benzyl carbamate optionally substituted by C₁-C₆ alkoxy, C₁-C₆ alkyl, halide, or an amino protecting group.

In certain embodiments, the novel intermediate compounds are separated into enantiomers, and a novel intermediate compound is:

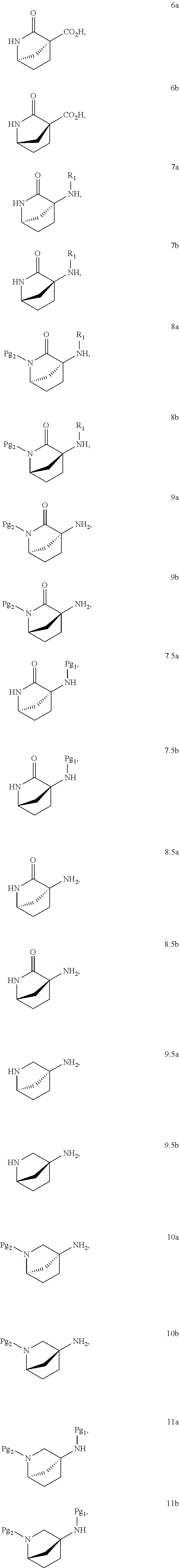

or a salt thereof.

### Certain Nonlimiting Embodiments

Certain aspects of the present invention are provided in the following numbered embodiments.
1. A method of preparing a compound of Formula (T-2):

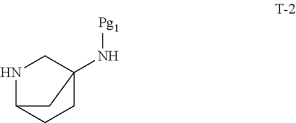

or a salt thereof, comprising:
reducing a compound of Formula (11):

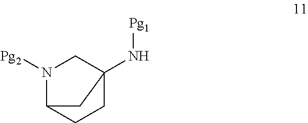

or a salt thereof,
wherein Pg₁ is a first protecting group; and
Pg₂ is a second protecting group;
to form the compound of Formula (T-2).
2. The method of embodiment 1, wherein:
   a) the compound of Formula (T-2) is the compound of Formula (T-3):

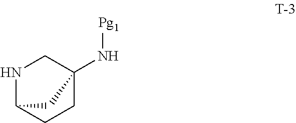

and the compound of Formula (11) is the compound of Formula (11a):

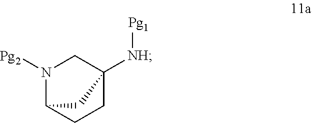

or
   b) compound of Formula (T-2) is the compound of Formula (T-4):

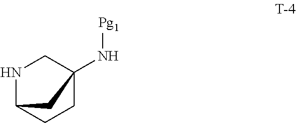

and the compound of Formula (11) is the compound of Formula (11b):

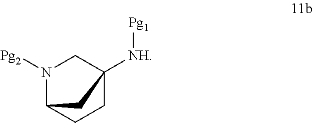

3. The method of embodiment 1 or embodiment 2, wherein Pg₁ and Pg₂ are each independently selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.
4. The method of embodiment 1 or embodiment 2, wherein Pg₁ is tert-butyloxycarbonyl.
5 The method of embodiment 1 or embodiment 2, wherein Pg₂ is a benzyl group optionally substituted by C₁-C₆ alkoxy, C₁-C₆ alkyl, or halide.
6. The method of any one of embodiments 1-5, wherein the reduction of Formula (11), or a salt thereof, is catalyzed by a transition metal to form the compound of Formula (T-2), or a salt thereof.
7 The method of embodiment 6, wherein the transition metal is palladium chloride, palladium on barium sulfate, or palladium on carbon (Pd/C).
8. The method of embodiment 6, wherein the transition metal is palladium on carbon (Pd/C).
9. The method of any one of embodiments 1-8, wherein the reaction is carried out using an alcohol as solvent.
10. The method of embodiment 9, wherein the alcohol is methanol, ethanol, or isopropanol.
11. The method of embodiment 9, wherein the alcohol is methanol.
12. The method of any one of embodiments 1-11, wherein the compound of Formula (11), or a salt thereof, is prepared by protecting the primary amine group of a compound of Formula (10):

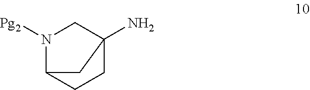

or a salt thereof.
13. A method of preparing a compound of Formula (11),

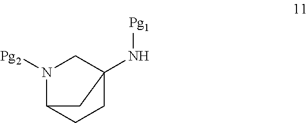

or a salt thereof, comprising:
protecting the primary amine group of a compound of Formula (10):

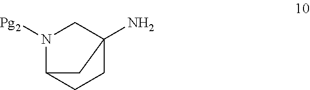

or a salt thereof, wherein:
wherein Pg₁ is a first protecting group; and
Pg₂ is a second protecting group;
to form the compound of Formula (11).
14. The method of embodiment 13, wherein Pg₁ and Pg₂ are each independently selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.
15. The method of embodiment 13, wherein Pg₁ is tert-butyloxycarbonyl.
16. The method of embodiment 13, wherein Pg₂ is a benzyl group optionally substituted by C₁-C₆ alkoxy, C₁-C₆ alkyl, or halide.
17. The method of any one of embodiments 12-16, wherein:
   a) the compound of Formula (11) is the compound of Formula (11a):

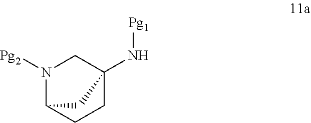

and the compound of Formula (10) is the compound of Formula (10a):

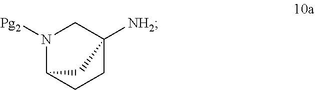

or
   b) the compound of Formula (11) is the compound of Formula (11b):

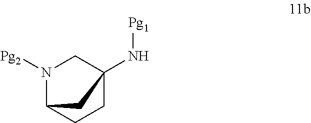

and the compound of Formula (10) is the compound of Formula (10b):

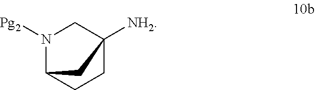

18. The method of any one of embodiments 12-17, wherein the reaction is carried out using an organic solvent.
19. The method of embodiment 18, wherein the organic solvent is chloroform, dichloroethane, toluene, methanol, ethanol, isopropanol, or 2-methyl-THF, dioxane, ethyl acetate, or tetrahydrofuran.
20. The method of embodiment 18, wherein the organic solvent is dichloromethane, methanol, toluene, or tetrahydrofuran.
21. The method of any one of embodiments 1-20, wherein the compound of Formula (10), or a salt thereof, is prepared by sequential protection of the substituent and heterocyclic amines of a compound of Formula (9.5):

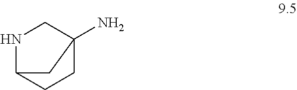

or a salt thereof, with Pg₂ and Pg₁, respectively, followed by selective deprotection of the substituent amine to form the compound of Formula (10).
22. The method of any one of embodiments 1-20, wherein the compound of Formula (10), or a salt thereof, is prepared by reducing a compound of Formula (9):

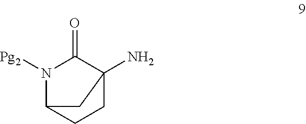

or a salt thereof, to form the compound of Formula (10).
23. A method of preparing a compound of Formula (10),

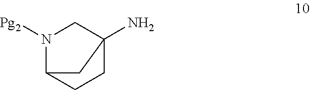

or a salt thereof, comprising:
reducing a compound of Formula (9):

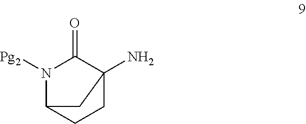

or a salt thereof,
wherein Pg₂ is a second protecting group, to form the compound of Formula (10).
24. The method of embodiment 23, wherein Pg₂ is selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.
25. The method of embodiment 23, wherein Pg₂ is a benzyl group optionally substituted by C₁-C₆ alkoxy, C₁-C₆ alkyl, or halide.
26. The method of any one of embodiments 22-25, wherein:
   a) the compound of Formula (10) is the compound of Formula (10a):

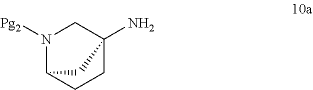

and the compound of Formula (9) is the compound of Formula (9a):

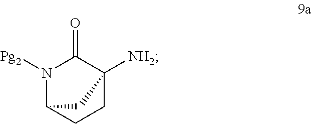

or
   b) the compound of Formula (10) is the compound of Formula (10b):

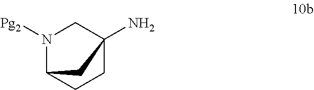

and the compound of Formula (9) is the compound of Formula (9b):

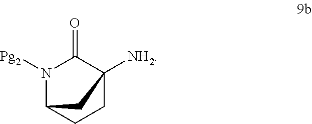

27. The method of any one of embodiments 22-26, wherein the reducing agent is a metal hydride, aluminum hydride, borohydride, or borane reagent.
28. The method of embodiment 27, wherein the reducing agent is lithium aluminum hydride, triphenylphosphine borane, borane tetrahydrofuran, or borane dimethylsulfide.
29. The method of embodiment 27, wherein the reducing agent is borane dimethylsulfide.
30. The method of any one of embodiments 22-29, wherein the reaction is carried out using an aprotic solvent.
31. The method of embodiment 30, wherein the aprotic solvent is diethyl ether, tetrahydrofuran, 2-methyltetrahydrofuran, acetonitrile, 1,4-dioxane, toluene, dichloromethane, dichloroethane, chloroform, or a mixture thereof.
32. The method of embodiment 30, wherein the aprotic solvent is tetrahydrofuran.
33. The method of any one of embodiments 22-32, wherein the reaction is carried out at a temperature of about 25-80 °C.
34. The method of embodiment 33, wherein the reaction is carried out at a temperature of about 60-70 °C.
35. The method of any one of embodiments 22-34, wherein the compound of Formula (9), or a salt thereof, is prepared by reducing a compound of Formula (8):

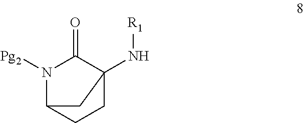

or a salt thereof, wherein:

R₁ is a benzyl carbamate optionally substituted by C₁-C₆ alkoxy, C₁-C₆ alkyl, halide, or an amino protecting group; and
Pg₂ is a second protecting group
to form the compound of Formula (9).
36. A method of preparing a compound of Formula (9),

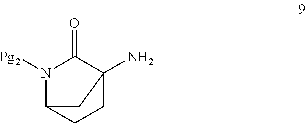

or a salt thereof, comprising:
reducing a compound of Formula (8):

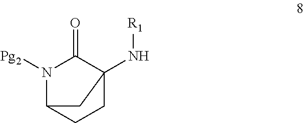

or a salt thereof, wherein:
R₁ is a benzyl carbamate optionally substituted by C₁-C₆ alkoxy, C₁-C₆ alkyl, halide, or an amino protecting group; and
Pg₂ is a second protecting group
to form a compound of Formula (9).
37. The method of embodiment 36, wherein Pg₂ is selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.
38. The method of embodiment 36, wherein Pg₂ is a benzyl group optionally substituted by C₁-C₆ alkoxy, C₁-C₆ alkyl, or halide.
39. The method of any one of embodiments 35-38, wherein:
   a) the compound of Formula (9) is the compound of Formula (9a):

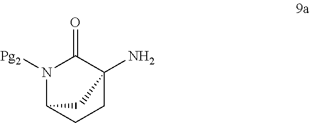

and the compound of Formula (8) is the compound of Formula (8a):

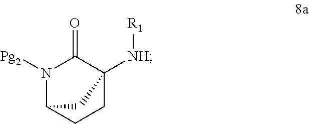

or
   b) the compound of Formula (9) is the compound of Formula (9b):

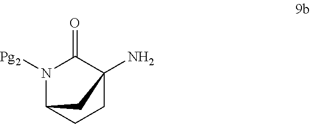

and the compound of Formula (8) is the compound of Formula (8b):

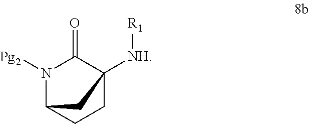

40. The method of any one of embodiments 35-39, wherein the reduction is catalyzed by a transition metal.
41. The method of embodiment 40, wherein the transition metal is palladium chloride, palladium on barium sulfate, or palladium on carbon (Pd/C).
42. The method of embodiment 40, wherein the transition metal is palladium on carbon (Pd/C).
43. The method of any one of embodiments 35-42, wherein the reaction is carried out using an alcohol as solvent.
44. The method of embodiment 43, wherein the alcohol is methanol, ethanol, or isopropanol.
45. The method of embodiment 43, wherein the alcohol is methanol.
46. The method of any one of embodiments 35-45, wherein the compound of Formula (8), or a salt thereof, is prepared by reacting a compound of Formula (7):

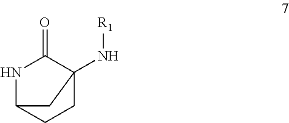

or a salt thereof, with Pg₂X, wherein:
R₁ is a benzyl carbamate optionally substituted by C₁-C₆ alkoxy, C₁-C₆ alkyl, halide, or an amino protecting group;
Pg₂ is a second protecting group; and
X is a leaving group;
to form the compound of Formula (8).
47. A method of preparing a compound of Formula (8),

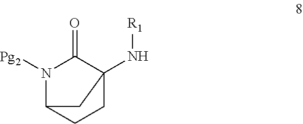

or a salt thereof, comprising:
reacting a compound of Formula (7):

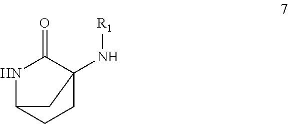

or a salt thereof, with Pg₂X, wherein:
R₁ is a benzyl carbamate optionally substituted by C₁-C₆ alkoxy, C₁-C₆ alkyl, halide, or an amino protecting group;
Pg₂ is a second protecting group; and
X is a leaving group;
to form the compound of Formula (8).
48. The method of embodiment 46 or embodiment 47, wherein:
   a) the compound of Formula (8) is the compound of Formula (8a):

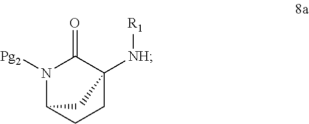

and the compound of Formula (7) is the compound of Formula (7a):

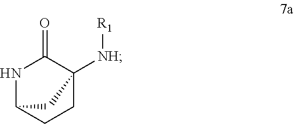

or
   b) the compound of Formula (8) is the compound of Formula (8b):

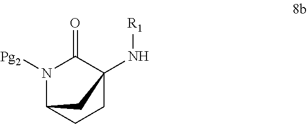

and the compound of Formula (7) is the compound of Formula (7b):

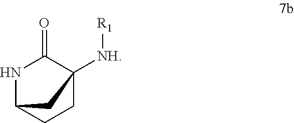

49. The method of any one of embodiments 46-48, wherein Pg₂ is selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.
50. The method of any one of embodiments 46-48, wherein Pg₂ is a benzyl group optionally substituted by C₁-C₆ alkoxy, C₁-C₆ alkyl, or halide
51. The method of any one of embodiments 46-50, wherein the reaction is carried out in the presence of a non-nucleophilic base.
52. The method of embodiment 51, wherein the non-nucleophilic base is N,N-diisopropylethylamine, lithium diisopropylamide, sodium bis(trimethylsilyl)amide, sodium tert-butoxide, or lithium bis(trimethylsilyl)amide.
53. The method of embodiment 51, wherein the non-nucleophilic base used in the reaction with the compound of Formula (7) is lithium bis(trimethylsilyl)amide.
54. The method of any one of embodiments 46-53, wherein the reaction is carried out using an ether solvent.
55. The method of embodiment 54, wherein the ether solvent is tetrahydrofuran, 2-methyltetrahydrofuran, 1,4-dioxane, methyl tert-butyl ether, or a mixture thereof.
56. The method of embodiment 54, wherein the ether solvent is tetrahydrofuran.
57. A method of preparing a compound of Formula (10),

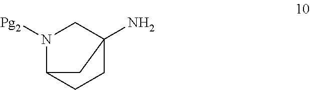

or a salt thereof, wherein Pg₂ is a second protecting group, comprising:
sequential protection of the substituent and heterocyclic amines of a compound of Formula (9.5):

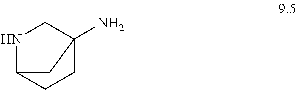

or a salt thereof, with Pg₂ and Pg₁, respectively, followed by selective deprotection of the substituent amine to form the compound of Formula (10).
58. The method of embodiment 57, wherein Pg₁ and Pg₂ are each independently selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.
59. The method of embodiment 57, wherein Pg₁ is optionally substituted benzyl.
60. The method of embodiment 57, wherein Pg₂ is an optionally substituted carboxybenzyl.
61. The method of any one of embodiments 21 or 57-60, wherein:
   a) the compound of Formula (10) is the compound of Formula (10a):

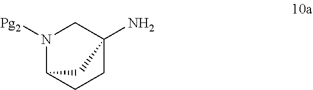

and the compound of Formula (9.5) is the compound of Formula (9.5a):

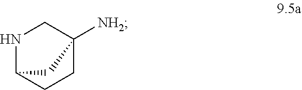

or
   b) the compound of Formula (10) is the compound of Formula (10b):

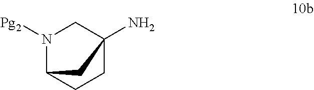

and the compound of Formula (9.5) is the compound of Formula (9.5b):

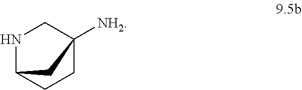

62. The method of any one of embodiments 21 or 57-61, wherein the protection reactions are carried out using an organic solvent.
63. The method of embodiment 62, wherein the organic solvent is chloroform, dichloromethane, tetrachloromethane, dichloroethane, toluene, methanol, ethanol, isopropanol, toluene, or tetrahydrofuran.
64. The method of embodiment 62, wherein the organic solvent is dichloromethane, methanol, toluene, or tetrahydrofuran.
65. The method of any one of embodiments 21 or 57-64, wherein the selective deprotection of the substituent amine is carried out with an acid.
66. The method of embodiment 65, wherein the acid is hydrochloric acid.
67. The method of any one of embodiments 21 or 57-66, wherein the compound of Formula (9.5), or a salt thereof, is prepared by reducing a compound of Formula (8.5),

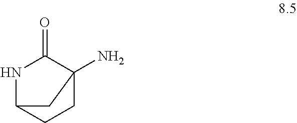

or a salt thereof, to form the compound of Formula (9.5).
68. A method of preparing a compound of Formula (9.5),

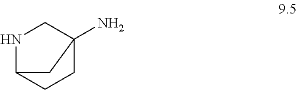

or a salt thereof, comprising:
reducing a compound of Formula (8.5):

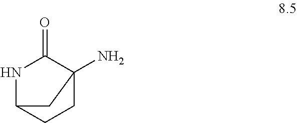

or a salt thereof, to form a compound of Formula (9.5).
69. The method of embodiment 67 or embodiment 68, wherein:
   a) the compound of Formula (9.5) is the compound of Formula (9.5a):

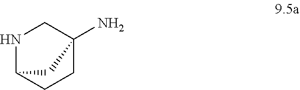

and the compound of Formula (8.5) is the compound of Formula (8.5a):

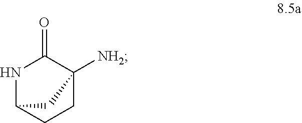

or
   c) the compound of Formula (9.5) is the compound of Formula (9.5b):

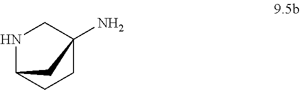

and the compound of Formula (8.5) is the compound of Formula (8.5b):

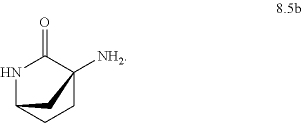

70. The method of any one of embodiments 67-69, wherein the reducing agent is a metal hydride, aluminum hydride, borohydride, or borane reagent.
71. The method of embodiment 70, wherein the reducing agent is lithium aluminum hydride, triphenylphosphine borane, borane tetrahydrofuran, or borane dimethylsulfide.
72. The method of embodiment 70, wherein the reducing agent is lithium aluminum hydride.
73. The method of any one of embodiments 67-72, wherein the reaction is carried out using an aprotic solvent.
74. The method of embodiment 73, wherein the aprotic solvent is diethyl ether, tetrahydrofuran, 2-methyltetrahydrofuran, acetonitrile, 1,4-dioxane, toluene, dichloromethane, dichloroethane, chloroform, or a mixture thereof.
75. The method of embodiment 73, wherein the aprotic solvent is diethyl ether, tetrahydrofuran or a mixture thereof.
76. The method of any one of embodiments 67-75, wherein the compound of Formula (8.5), or a salt thereof, is prepared by hydrolyzing a compound of Formula (7.5):

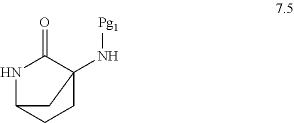

or a salt thereof, wherein Pg₁ is a first protecting group to form a compound of Formula (8.5).
77. A method of preparing a compound of Formula (8.5),

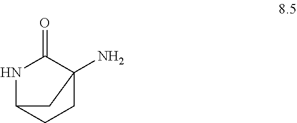

or a salt thereof, comprising:
hydrolyzing a compound of Formula (7.5):

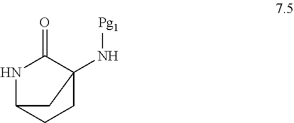

or a salt thereof, wherein Pg₁ is a first protecting group to form a compound of Formula (8.5).
78. The method of embodiment 76 or embodiment 77, wherein Pg₁ is selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.
79. The method of embodiment 76 or embodiment 77, wherein Pg₁ is tert-butyloxycarbonyl.
80. The method of any one of embodiments 76-79, wherein:
   a) the compound of Formula (8.5) is the compound of Formula (8.5a):

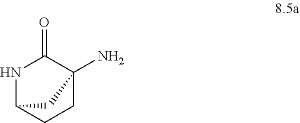

and the compound of Formula (7) is the compound of Formula (7a):

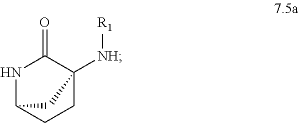

or
   d) the compound of Formula (8.5) is the compound of Formula (8.5b):

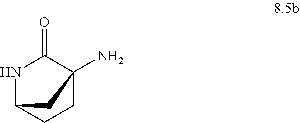

and the compound of Formula (7.5) is the compound of Formula (7.5b):

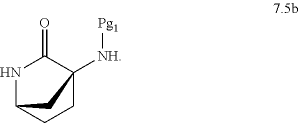

81. The method of any one of embodiments 76-80, wherein the hydrolysis is carried out with an acid.
82. The method of embodiment 81, wherein the acid is trifluoroacetic acid, hydrochloride, or phosphoric acid.
83. The method of embodiment 81, wherein the acid is trifluoroacetic acid.
84. The method of any one of embodiments 76-83, wherein the protection reactions are carried out using an organic solvent.
85. The method of embodiment 84, wherein the organic solvent is chloroform, dichloromethane, tetrachloromethane, dichloroethane, toluene, methanol, ethanol, isopropanol, toluene, or tetrahydrofuran.
86. The method of embodiment 84, wherein the organic solvent is dichloromethane, methanol, or toluene.
87. The method of any one of embodiments 76-86, wherein the reaction is carried out at a temperature of about 20-30 °C.
88. The method of any one of embodiments 76-87, wherein the compound of Formula (7.5), or a salt thereof, is prepared by deprotecting and subsequently protecting the primary amine group of a compound of Formula (7),

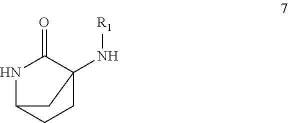

or a salt thereof.
89. A method of preparing a compound of Formula (7.5),

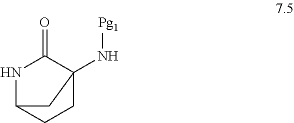

or a salt thereof, wherein Pg₁ is a first protecting group, wherein the method comprises: deprotecting and subsequently protecting the primary amine group of a compound of Formula (7),

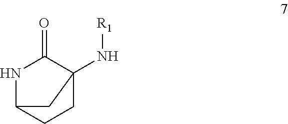

or a salt thereof, wherein R₁ is a benzyl carbamate optionally substituted by C₁-C₆ alkoxy, C₁-C₆ alkyl, halide, or an amino protecting group, to form the compound of Formula (7.5).
90. The method of embodiment 88 or embodiment 89, wherein Pg₁ is selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.
91. The method of embodiment 88 or embodiment 89, wherein Pg₁ is tert-butyloxycarbonyl.
92. The method of any one of embodiments 88-91, wherein:
   a) the compound of Formula (7.5) is the compound of Formula (7.5a):

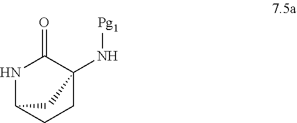

and the compound of Formula (7) is the compound of Formula (7a):

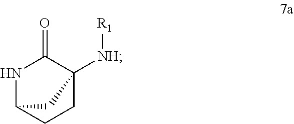

or
   b) the compound of Formula (7.5) is the compound of Formula (7.5b):

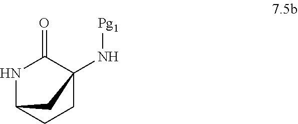

and the compound of Formula (7) is the compound of Formula (7b):

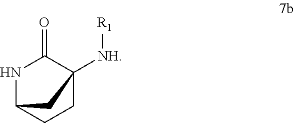

93. The method of any one of embodiments 88-92, wherein the deprotection is catalyzed by a transition metal.
94. The method of embodiment 93, wherein the transition metal is palladium chloride, palladium on barium sulfate, or palladium on carbon (Pd/C).
95. The method of embodiment 93, wherein the transition metal is palladium on carbon (Pd/C).
96. The method of any one of embodiments 88-95, wherein the reaction is carried out using an alcohol as solvent.
97. The method of embodiment 96, wherein the alcohol is methanol, ethanol, or isopropanol.
98. The method of embodiment 96, wherein the alcohol is methanol.
99. The method of any one of embodiments 88-98, wherein the protection reaction is carried out using an organic solvent.
100. The method of embodiment 99, wherein the organic solvent is chloroform, dichloromethane, tetrachloromethane, dichloroethane, toluene, methanol, ethanol, isopropanol, toluene, or tetrahydrofuran.
101. The method of embodiment 99, wherein the organic solvent is dichloromethane, methanol, toluene, or tetrahydrofuran.
102. The method of any one of embodiments 46-56 or 88-101, wherein the compound of Formula (7), or a salt thereof, is prepared by reacting a compound of Formula (6):

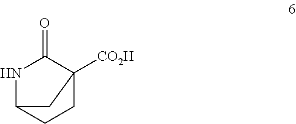 [SMILES: O=C(O)C12CCC(C1)NC2=O]

or a salt thereof,
with R₃OH, wherein R₃ is benzyl optionally substituted by C₁-C₆ alkoxy, C₁-C₆ alkyl, or halide, in the presence of diphenylphosphoryl azide and an amine base, to form the compound of Formula (7).
103. A method of preparing a compound of Formula (7),

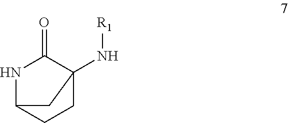

or a salt thereof, wherein R₁ is a benzyl carbamate optionally substituted by C₁-C₆ alkoxy, C₁-C₆ alkyl, halide, or an amino protecting group, comprising:
reacting a compound of Formula (6):

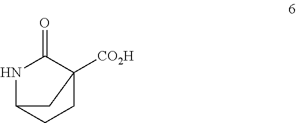 [SMILES: O=C(O)C12CCC(C1)NC2=O]

or a salt thereof, with R₃OH, wherein R₃ is a benzyl optionally substituted by C₁-C₆ alkoxy, C₁-C₆ alkyl, or halide, in the presence of diphenylphosphoryl azide and an amine base, to form the compound of Formula (7).
104. The method of embodiment 102 or embodiment 103, wherein:
   a) the compound of Formula (7) is the compound of Formula (7a):

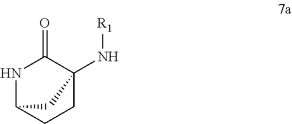

and the compound of Formula (6) is the compound of Formula (6a):

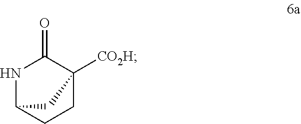 [SMILES: O=C(O)[C@]12CC[C@H](C1)NC2=O]

or
   b) the compound of Formula (7) is the compound of Formula (7b):

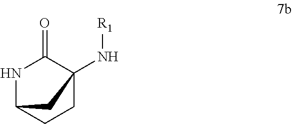

and the compound of Formula (6) is the compound of Formula (6b):

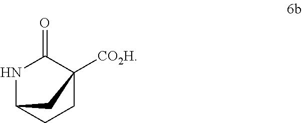 [SMILES: O=C(O)[C@@]12CC[C@@H](C1)NC2=O]

105. The method of any one of embodiments 102-104, wherein the amine base is 2,2,6,6-tetramethylpiperidine, trimethylamine, triethylamine, or N,N-diisoproypylethylamine.
106. The method of embodiment 105, wherein the amine base is N,N-diisoproypylethylamine.
107. The method of any one of embodiments 102-106, wherein the reaction is carried out using an organic solvent.
108. The method of embodiment 107, wherein the organic solvent used in the reaction is dimethyl sulfoxide, dichloromethane, tetrahydrofuran, 2-methyltetrahydrofuran, or a mixture thereof.
109. The method of embodiment 107, wherein the organic solvent used in the reaction is 2-methyltetrahydrofuran.
110. The method of any one of embodiments 102-109, wherein the reaction is carried out at a starting temperature of about 40-70 °C, then at a reflux temperature above 50-120 °C.
111. The method of embodiment 110, wherein the reaction is carried out at a starting temperature of about 50-60 °C or about 60-70 °C, then at a reflux temperature of about 70-100 °C.
112. The method of any one of embodiments 102-111, wherein the compound of Formula (7), or a salt thereof, is separated into enantiomers, a compound of Formula (7a) and a compound of Formula (7b), or salts thereof, using super fluid chromatography.
113. The method of any one of embodiments 102-112, wherein the compound of Formula (6), or a salt thereof, is prepared by reacting a compound of Formula (5):

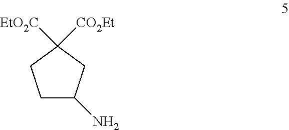 [SMILES: CCOC(=O)C1(C(=O)OCC)CCC(N)C1]

or a salt thereof, with a base to form the compound of Formula (6).
114. A method of preparing a compound of Formula (6),

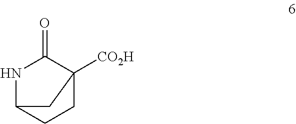 [SMILES: O=C(O)C12CCC(C1)NC2=O]

or a salt thereof, comprising:
reacting a compound of Formula (5):

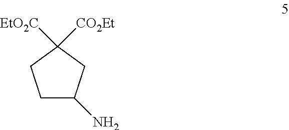 [SMILES: CCOC(=O)C1(C(=O)OCC)CCC(N)C1]

or a salt thereof, with a base to form the compound of Formula (6).
115. The method of embodiment 113 or 114, wherein:
   a) the compound of Formula (6) is a compound of Formula (6a):

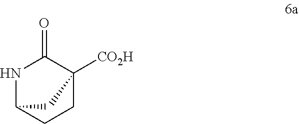 [SMILES: O=C(O)[C@]12CC[C@H](C1)NC2=O]

and the compound of Formula (5) is a compound of Formula (5a),

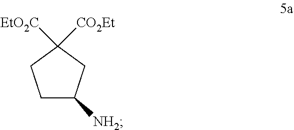 [SMILES: CCOC(=O)C1(C(=O)OCC)CC[C@H](N)C1]

or
   b) the compound of Formula (6) is a compound of Formula (6b):

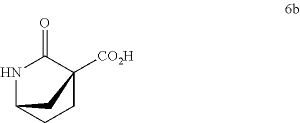 [SMILES: O=C(O)[C@@]12CC[C@@H](C1)NC2=O]

and the compound of Formula (5) is a compound of Formula (5b),

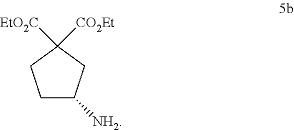 [SMILES: CCOC(=O)C1(C(=O)OCC)CC[C@@H](N)C1]

116. The method of any one of embodiments 113-115, wherein the base is an inorganic base.
117. The method of embodiment 116, wherein the inorganic base is sodium tert-butoxide, potassium tert-butoxide, potassium carbonate, or cesium carbonate.
118. The method of embodiment 116, wherein the inorganic base is cesium carbonate.
119. The method of any one of embodiments 113-118, wherein the reaction is carried out using an alcohol as solvent.
120. The method of embodiment 119, wherein the alcohol is methanol, ethanol, or isopropanol.
121. The method of embodiment 119, wherein the alcohol is methanol.
122. The method of any one of embodiments 113-121, wherein the reaction is carried out at a temperature of about 25-80 °C.
123. The method of embodiment 122, wherein the reaction is carried out at a temperature of about 50-70 °C.
124. The method of any one of embodiments 113-123, wherein the compound of Formula (5), or a salt thereof, is prepared by deprotecting the amine of a compound of Formula (4):

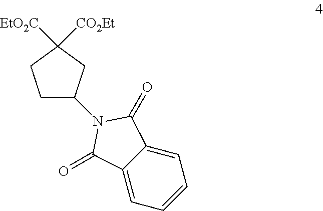 [SMILES: CCOC(=O)C1(C(=O)OCC)CCC(N2C(=O)c3ccccc3C2=O)C1]

or a salt thereof, to form the compound of Formula (5).
125. A method of preparing a compound of Formula (5),

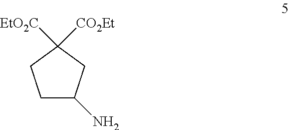 [SMILES: CCOC(=O)C1(C(=O)OCC)CCC(N)C1]

or a salt thereof, comprising:
deprotecting a compound of Formula (4):

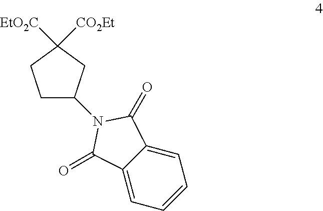 [SMILES: CCOC(=O)C1(C(=O)OCC)CCC(N2C(=O)c3ccccc3C2=O)C1]

or a salt thereof, to form the compound of Formula (5).
126. The method of embodiment 124 or embodiment 125, wherein:
   a) the compound of Formula (5) is a compound of Formula (5a):

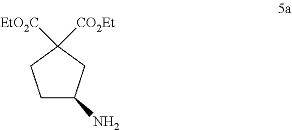 [SMILES: CCOC(=O)C1(C(=O)OCC)CC[C@H](N)C1]

and the compound of Formula (4) is a compound of Formula (4a),

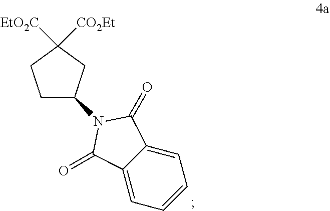 [SMILES: CCOC(=O)C1(C(=O)OCC)CC[C@H](N2C(=O)c3ccccc3C2=O)C1]

or
   b) the compound of Formula (5) is a compound of Formula (5b):

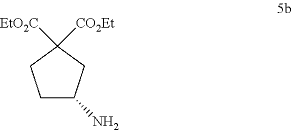 [SMILES: CCOC(=O)C1(C(=O)OCC)CC[C@@H](N)C1]

and the compound of Formula (4) is a compound of Formula (4b),

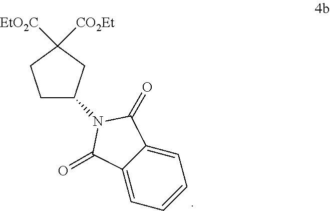 [SMILES: CCOC(=O)C1(C(=O)OCC)CC[C@@H](N2C(=O)c3ccccc3C2=O)C1]

127. The method of any one of embodiments 124-126, wherein the deprotection is carried out using a base.
128. The method of embodiment 127, wherein the base is sodium borohydride, methylamine, methylhydrazine, or hydrazine.
129. The method of embodiment 127, wherein the base is hydrazine.
130. The method of any one of embodiments 124-129, wherein the reaction is carried out using a mixture of alcohol and an organic solvent.
131. The method of embodiment 130, wherein the alcohol is methanol, ethanol, or isopropanol.
132. The method of embodiment 130, wherein the organic solvent is dimethyl sulfoxide, dichloromethane, tetrahydrofuran, or 2-methyltetrahydrofuran.
133. The method of embodiment 130, wherein the alcohol is methanol, and the organic solvent is dichloromethane.
134. The method of any one of embodiments 124-133, wherein the compound of Formula (4), or a salt thereof, is prepared by reacting a compound of Formula (3):

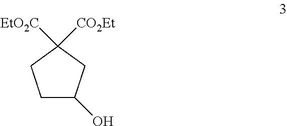 [SMILES: CCOC(=O)C1(C(=O)OCC)CCC(O)C1]

or a salt thereof, with phthalimide to form the compound of Formula (4).
135. A method of preparing a compound of Formula (4),

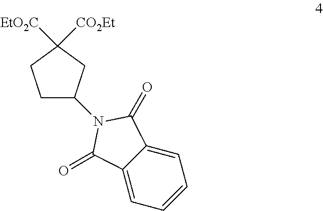 [SMILES: CCOC(=O)C1(C(=O)OCC)CCC(N2C(=O)c3ccccc3C2=O)C1]

or a salt thereof, comprising,
reacting a compound of Formula (3):

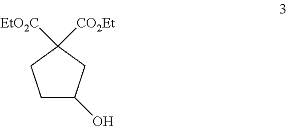 [SMILES: CCOC(=O)C1(C(=O)OCC)CCC(O)C1]

or a salt thereof, with phthalimide to form the compound of Formula (4).
136. The method of embodiment 134 or embodiment 135, wherein:
   a) the compound of Formula (4) is a compound of Formula (4a):

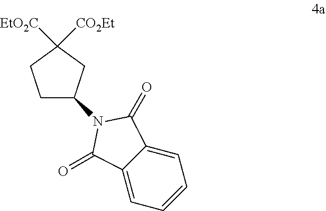 [SMILES: CCOC(=O)C1(C(=O)OCC)CC[C@H](N2C(=O)c3ccccc3C2=O)C1]

and the compound of Formula (3) is the compound of Formula (3a),

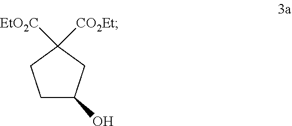 [SMILES: CCOC(=O)C1(C(=O)OCC)CC[C@H](O)C1]

or
   b) the compound of Formula (4) is a compound of Formula (4b):

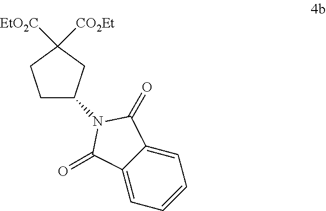 [SMILES: CCOC(=O)C1(C(=O)OCC)CC[C@@H](N2C(=O)c3ccccc3C2=O)C1]

and the compound of Formula (3) is a compound of Formula (3b),

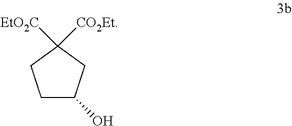 [SMILES: CCOC(=O)C1(C(=O)OCC)CC[C@@H](O)C1]

137. The method of any one of embodiments 134-136, wherein the reaction further comprises triphenylphosphine and diisopropyl azodicarboxylate.
138. The method of any one of embodiments 134-137, where in the reaction is carried out using an aprotic solvent.
139. The method of embodiment 138, wherein the aprotic solvent is diethyl ether, tetrahydrofuran, 2-methyltetrahydrofuran, acetonitrile, 1,4-dioxane, toluene, dichloromethane, dichloroethane, chloroform, or a mixture thereof.
140. The method of embodiment 138, wherein the aprotic solvent is tetrahydrofuran.
141. The method of any one of embodiments 134-140, wherein the reaction is carried out at a temperature of about 0-30 °C.
142. The method of embodiment 141, wherein the reaction is carried out at a starting temperature of about 0-10 °C, then at 20-30 °C.
143. The method of any one of embodiments 134-142, wherein the compound of Formula (3), or a salt thereof, is prepared by reacting a compound of Formula (1):

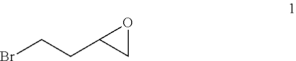

or a salt thereof,
and a compound of Formula (2):

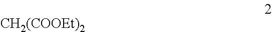

or a salt thereof, to form the compound of Formula (3).
144. A method of preparing a compound of Formula (3),

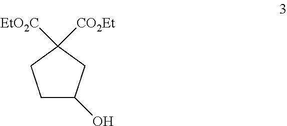 [SMILES: CCOC(=O)C1(C(=O)OCC)CCC(O)C1]

or a salt thereof, comprising:
reacting a compound of Formula (1):

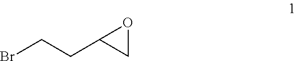

or a salt thereof,
and a compound of Formula (2):

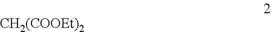

or a salt thereof, to form the compound of Formula (3).
145. The method of embodiment 143 or embodiment 144, wherein:
   a) the compound of Formula (3) is a compound of Formula (3a):

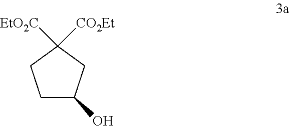 [SMILES: CCOC(=O)C1(C(=O)OCC)CC[C@H](O)C1]

and the compound of Formula (1) is a compound of Formula (1a),

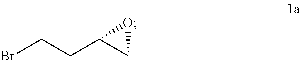

or
   b) the compound of Formula (3) is a compound of Formula (3b):

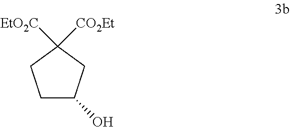 [SMILES: CCOC(=O)C1(C(=O)OCC)CC[C@@H](O)C1]

and the compound of Formula (1) is the compound of Formula (1b),

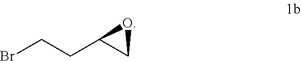

146. The method of any one of embodiments 143-145, wherein the reaction is carried out in the presence of a base.
147. The method of embodiment 146, wherein the base is sodium hydride, potassium tert-butoxide, or sodium ethoxide.
148. The method of embodiment 146, wherein the base is sodium ethoxide.
149. The method of any one of embodiments 143-148, wherein the reaction is carried out using an alcohol as solvent.
150. The method of embodiment 149, wherein the alcohol is methanol, ethanol, or isopropanol.
151. The method of embodiment 149, wherein the alcohol is ethanol.
152. The method of any one of embodiments 143-151, wherein the reaction is carried out at a temperature of about 0-30 °C.
153. The method of embodiment 152, wherein the reaction is carried out at a starting temperature of about 0-10 °C, then at 20-30 °C.
154. The method of any one of embodiments 143-153, wherein the compound of Formula (1), or a salt thereof, is separated into enantiomers, Formula (1a) and Formula (1b), or salts thereof, using kinetic resolution.
155. The method of embodiment 154, wherein the kinetic resolution is Jacobsen's kinetic resolution or hydrolytic kinetic resolution catalyzed by chiral cobalt-salen complexes.
156. A method of preparing a compound of Formula (T-2), or a salt thereof, comprising the following steps:

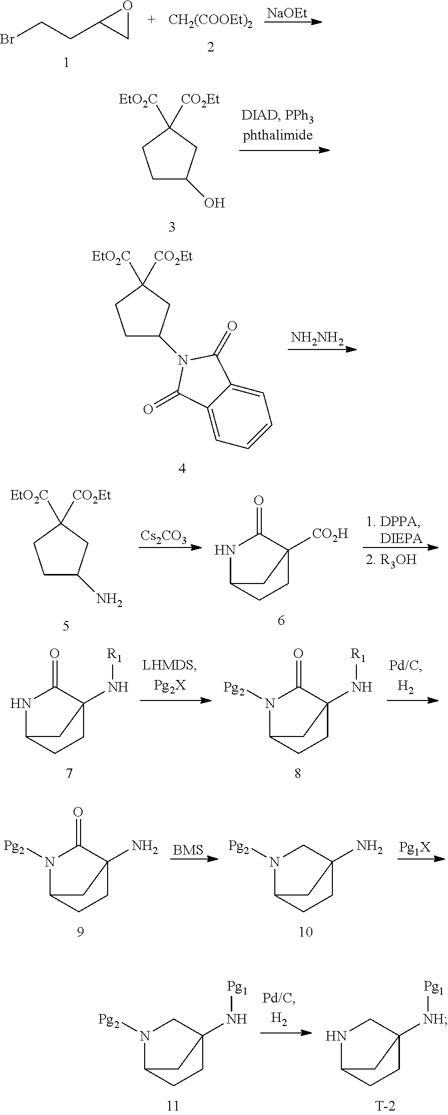

wherein:
R₃ is benzyl optionally substituted by C₁-C₆ alkoxy, C₁-C₆ alkyl, or halide;
R₁ is benzyl carbamate optionally substituted by C₁-C₆ alkoxy, C₁-C₆ alkyl, or halide;
Pg₁ is a first protecting group;
Pg₂ is a second protecting group; and
X is a leaving group.
157. A method of preparing a compound of Formula (T-2), or a salt thereof, comprising the following steps:

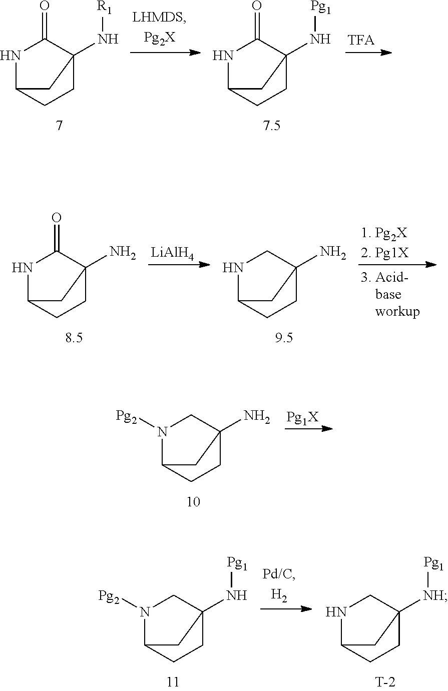

wherein:
R₁ is benzyl carbamate optionally substituted by C₁-C₆ alkoxy, C₁-C₆ alkyl, or halide;
Pg₁ is a first protecting group;
Pg₂ is a second protecting group; and
X is a leaving group.
158. The method of embodiment 156 or embodiment 157, wherein Pg₁ and Pg₂ are each independently selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.
159. A compound of Formula (T-3):

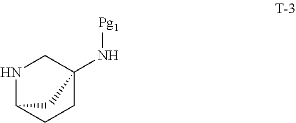

or a salt thereof, wherein Pg₁ is a first protecting group.
160. A compound of Formula (T-4):

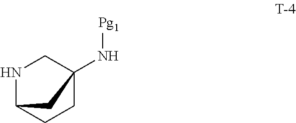

or a salt thereof, wherein Pg₁ is a first protecting group.
161. The compound of embodiment 159 or embodiment 160, wherein Pg₁ is selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.
162. The compound of embodiment 159 or embodiment 160, wherein Pg₁ is tert-butyloxycarbonyl.
163. A compound of Formula (3):

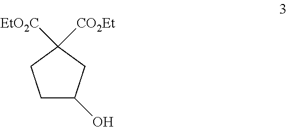 [SMILES: CCOC(=O)C1(C(=O)OCC)CCC(O)C1]

or a salt thereof.
164. The compound of embodiment 163, wherein the compound of Formula (3), or a salt thereof, is a compound of Formula (3a):

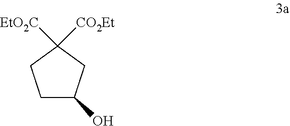 [SMILES: CCOC(=O)C1(C(=O)OCC)CC[C@H](O)C1]

or a salt thereof; or a compound of Formula (3b):

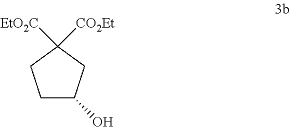 [SMILES: CCOC(=O)C1(C(=O)OCC)CC[C@@H](O)C1]

or a salt thereof.
165. A compound of Formula (4):

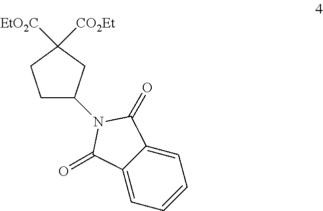 [SMILES: CCOC(=O)C1(C(=O)OCC)CCC(N2C(=O)c3ccccc3C2=O)C1]

or a salt thereof.
166. The compound of embodiment 165, wherein the compound of Formula (4), or a salt thereof, is a compound of Formula (4a):

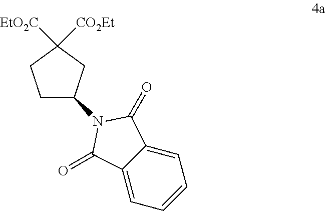 [SMILES: CCOC(=O)C1(C(=O)OCC)CC[C@H](N2C(=O)c3ccccc3C2=O)C1]

or a salt thereof; or a compound of Formula (4b):

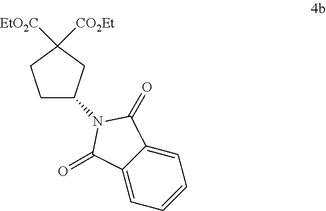 [SMILES: CCOC(=O)C1(C(=O)OCC)CC[C@@H](N2C(=O)c3ccccc3C2=O)C1]

or a salt thereof.
167. A compound of Formula (5):

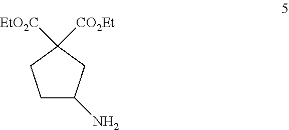 [SMILES: CCOC(=O)C1(C(=O)OCC)CCC(N)C1]

or a salt thereof.
168. The compound of embodiment 167, wherein the compound of Formula (5), or a salt thereof, is a compound of Formula (5a):

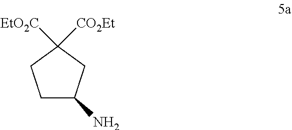 [SMILES: CCOC(=O)C1(C(=O)OCC)CC[C@H](N)C1]

or a salt thereof; or a compound of Formula (5b):

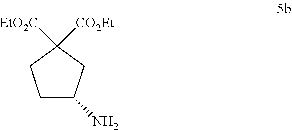 [SMILES: CCOC(=O)C1(C(=O)OCC)CC[C@@H](N)C1]

or a salt thereof.
169. A compound of Formula (6):

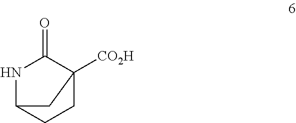 [SMILES: O=C(O)C12CCC(C1)NC2=O]

or a salt thereof.
170. The compound of embodiment 169, wherein the compound of Formula (6), or a salt thereof, is a compound of Formula (6a):

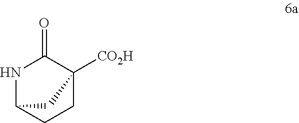 [SMILES: O=C(O)[C@]12CC[C@H](C1)NC2=O]

or a salt thereof; or a compound of Formula (6b):

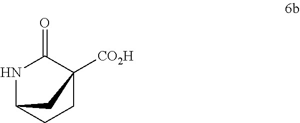 [SMILES: O=C(O)[C@@]12CC[C@@H](C1)NC2=O]

or a salt thereof.
171. A compound of Formula (7):

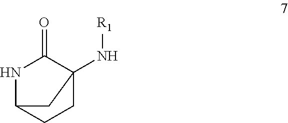

or a salt thereof, wherein R₁ is a benzyl carbamate optionally substituted by C₁-C₆ alkoxy, C₁-C₆ alkyl, or halide, or an amino protecting group.
172. The compound of embodiment 171, wherein the compound of Formula (7), or a salt thereof, is a compound of Formula (7a):

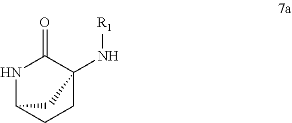

or a salt thereof; or a compound of Formula (7b):

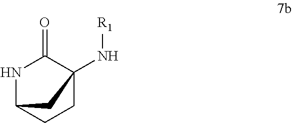

or a salt thereof.
173. A compound of Formula (8):

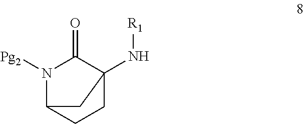

or a salt thereof, wherein:

R₁ is a benzyl carbamate optionally substituted by C₁-C₆ alkoxy, C₁-C₆ alkyl, halide, or an amino protecting group; and
Pg₂ is a second protecting group.
174. The compound of embodiment 173, wherein the compound of Formula (8), or a salt thereof, is a compound of Formula (8a):

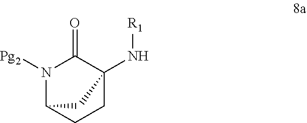

or a salt thereof; or a compound of Formula (8b):

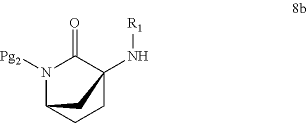

or a salt thereof.
175. A compound of Formula (9):

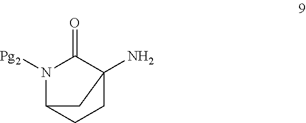

or a salt thereof, wherein Pg₂ is a second protecting group.
176. The compound of embodiment 175, wherein the compound of Formula (9), or a salt thereof, is a compound of Formula (9a):

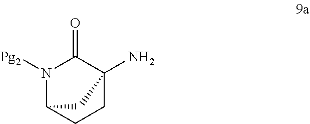

or a salt thereof; or a compound of Formula (9b):

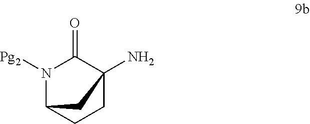

or a salt thereof.
177. A compound of Formula (7.5):

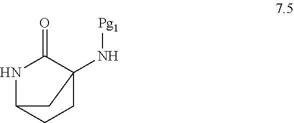

or a salt thereof, wherein Pg₁ is a first protecting group.
178. The compound of embodiment 177, wherein the compound of Formula (7.5), or a salt thereof, is a compound of Formula (7.5a):

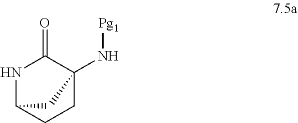

or a salt thereof; or a compound of Formula (7.5b):

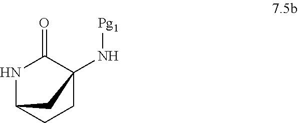

or a salt thereof.
179. A compound of Formula (8.5):

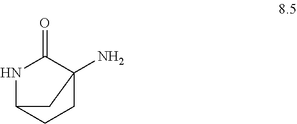

or a salt thereof.
180. The compound of embodiment 179, wherein the compound of Formula (8.5), or a salt thereof, is a compound of Formula (8.5a):

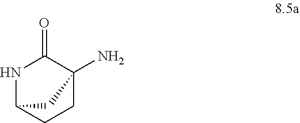

or a salt thereof; or a compound of Formula (8.5b):

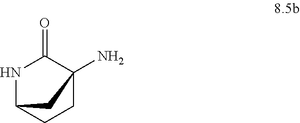

or a salt thereof.
181. A compound of Formula (9.5):

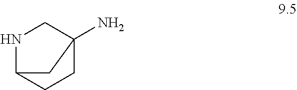

or a salt thereof.
182. The compound of embodiment 181, wherein the compound of Formula (9.5), or a salt thereof, is a compound of Formula (9.5a):

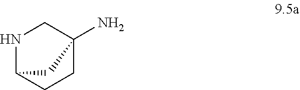

or a salt thereof; or a compound of Formula (9.5b):

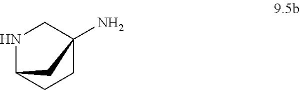

or a salt thereof.
183. A compound of Formula (10):

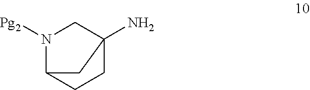

or a salt thereof, wherein Pg₂ is a second protecting group.
184. The compound of embodiment 183, wherein the compound of Formula (10), or a salt thereof, is a compound of Formula (10a):

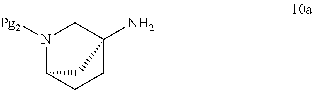

or a salt thereof; or a compound of Formula (10b):

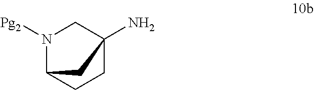

or a salt thereof.
185. A compound of Formula (11):

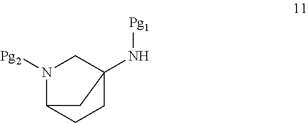

or a salt thereof, wherein:
Pg₁ is a first protecting group; and
Pg₂ is a second protecting group.
186. The compound of embodiment 185, wherein the compound of Formula (11), or a salt thereof, is a compound of Formula (11a):

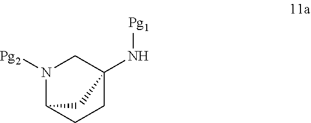

or a salt thereof; or a compound of Formula (11b):

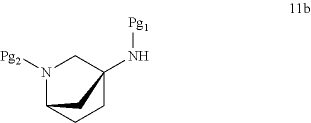

or a salt thereof.
187. The compound of embodiment 185 or embodiment 186, wherein Pg₁ is selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.
188. The compound of embodiment 185 or embodiment 186, wherein Pg₁ is tert-butyloxycarbonyl.
189. The compound of any one of embodiments 233-242, wherein Pg₂ is selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.
190. The compound of any one of embodiments 173-176 or 183-189, wherein Pg₂ is a benzyl group optionally substituted by C₁-C₆ alkoxy, C₁-C₆ alkyl, or halide.

### Examples

These examples are provided for illustrative purposes only and not to limit the scope of the claims provided herein.

Abbreviations used:

| | |
|---|---|
| 2-MeTHF | 2-methyltetrahydrofuran |
| BMS | borane dimethyl sulfide |
| Cbz-OSu | N-(benzyloxycarbonyloxy)succinimide |
| DCM | dichloromethane |
| DIAD | diisopropyl azodicarboxylate |
| DIEPA | N,N-diisopropylethylamine |
| DMSO | dimethyl sulfoxide |
| DPPA | diphenyl phosphorazidate |
| EtOAc | ethyl acetate |
| EtOH | ethanol |
| h | hour(s) |
| LC-MS, LC/MS, LCMS | liquid chromatography-mass spectrometry |
| LHMDS | lithium hexamethyldisilazide |
| MeOH | methanol |
| min | minute(s) |
| MTBE | methyl tert-butyl ether |
| NaOEt | sodium ethoxide |
| NMR | nuclear magnetic resonance |
| SFC | supercritical fluid chromatography |
| TFA | trifluoroacetic acid |
| THF | tetrahydrofuran |

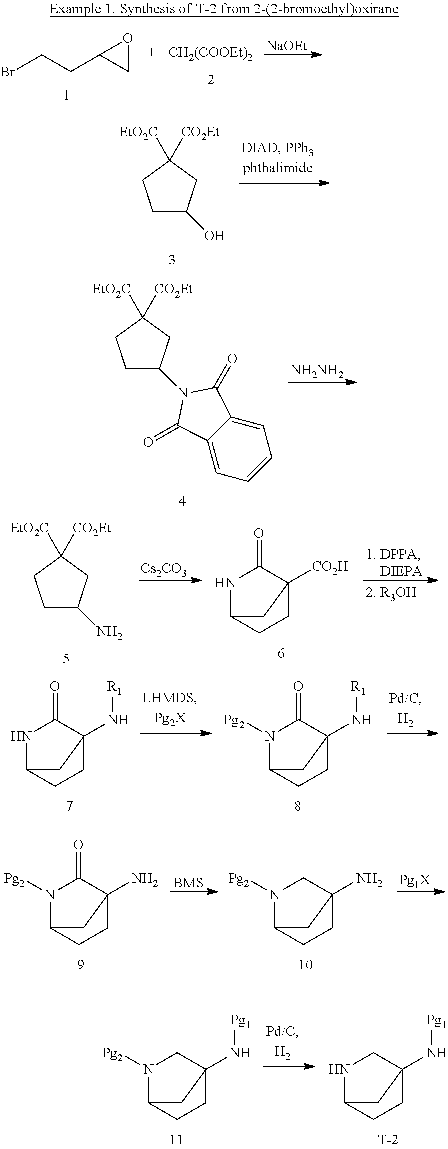

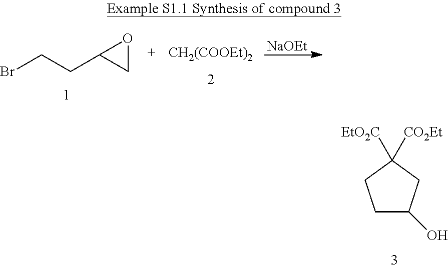 [SMILES: BrCCC1CO1 ; CCOC(=O)C1(C(=O)OCC)CCC(O)C1 ; "CH2(COOEt)2" ; "NaOEt"]

To a 3 L 3-neck round bottom flask was added diethyl malonate (156 g, 964 mmol) and EtOH (740 mL). The solution was cooled to 0-5 °C. A brown solution of sodium ethoxide (396 mL, 21 wt % in EtOH, 1060 mmol) was added through an additional funnel over 20 min. The temperature was then slightly increased to 9 °C. After stirring the reaction mixture at 0-5 °C for 15 min, 2-(2-bromoethyl) oxirane (150 g, 964 mmol) was added, and the reaction mixture was stirred at room temperature to give a brown suspension. After stirring for 18 h at room temperature, the reaction mixture was concentrated in vacuo and the residue was diluted with EtOAc (1 L) and brine (500 mL). The aqueous layer was extracted with EtOAc (300 mL) again. The combined organic layer was separated, dried over Na₂SO₄, filtered and concentrated in vacuo to give the product diethyl 3-hydroxycyclopentane-1,1-dicarboxylate (200 g, 782 mmol, 81% yield) as a brown oil with ~90% purity, which was used directly for next step without further purification.

LC-MS [M+1] 231.

¹H NMR (400 MHz, CHLOROFORM-d) δ 4.44-4.36 (m, 1H), 4.26-4.15 (m, 4H), 2.47-2.26 (m, 3H), 2.07 (br d, J=4.5 Hz, 1H), 1.94 (dtd, J=13.5, 8.5, 5.2 Hz, 1H), 1.84-1.67 (m, 1H), 1.33-1.20 (m, 6H).

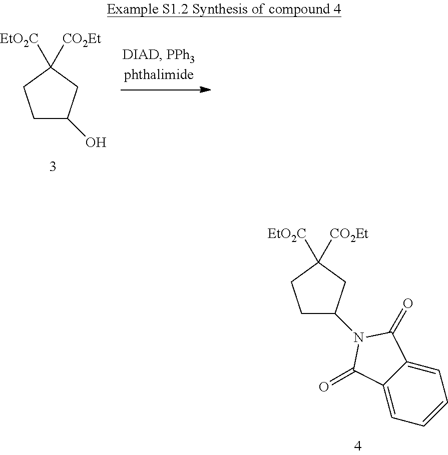 [SMILES: CCOC(=O)C1(C(=O)OCC)CCC(O)C1 ; CCOC(=O)C1(C(=O)OCC)CCC(N2C(=O)c3ccccc3C2=O)C1]

To a 5 L 3-neck round bottom 53orti was added triphenylphosphine (PPh₃, 117 g, 443 mmol) and THF (1400 mL). The colorless solution was cooled to 5 °C with an ice-water bath, and diisopropyl azodicarboxylate (DIAD, 86 mL, 443 mmol) was added dropwise over 35 min through an additional funnel, while the internal temperature was kept below 15 °C. The yellow color of DIAD was allowed to almost disappear before each addition. The clear reaction mixture became a white suspension during the addition of DIAD. The white thick suspension was stirred in an ice-water bath for 30 min. Formation of a pure white thick slurry indicated successful preparation of a PPh₃-DIAD complex. A solution of diethyl 3-hydroxycyclopentane-1,1-dicarboxylate (108 g, 422 mmol) was poured into the reaction mixture and the temperature was increased to 15 °C. Isoindoline-1,3-dione (59.9 g, 407 mmol) was then added in small portions while keeping the reaction temperature below 15 °C. After addition, the reaction mixture was allowed to warm to room temperature. The reaction mixture became a light orange solution and was stirred at room temperature overnight. The reaction mixture was quenched with brine (400 mL) and the aqueous layer was separated and extracted with EtOAc (300 mL). The combined organic layers were dried over Na₂SO₄, filtered and concentrated in vacuo to 1 L. Precipitation of colorless crystals (triphenylphosphine oxide (Ph₃P═O)) from the concentrated EtOAc solution was observed. The suspension was allowed to sit at room temperature overnight. The filtrate brown oil was added into a stirring 500 mL Et₂O in a 2 L beaker which formed a suspension. After 30 min, the resulting peach color solid was removed by filtration. The Et₂O filtrate was filtered one more time and then concentrated to give a crude brown oil. It was added to MeOH (200 mL) to form a white suspension. The precipitated pure product was collected by filtration and washed with methanol to give a white solid. The mother liquid was concentrated and purified on ISCO column chromatography with a gradient 0-10% acetone/hexane. A total of 93 g (yield 61%) diethyl 3-(1,3-dioxoisoindolin-2-yl)cyclopentane-1,1-dicarboxylate was obtained as a white solid.

LC-MS [M+1] 360.0.

¹H NMR (400 MHz, CHLOROFORM-d) δ 7.83 (dd, J=5.5, 3.0 Hz, 2H), 7.73-7.69 (m, 2H), 4.81 (quin, J=8.8 Hz, 1H), 4.29-4.20 (m, 4H), 2.78-2.66 (m, 3H), 2.36-2.28 (m, 1H), 2.19 (dt, J=13.3, 7.5 Hz, 1H), 2.11-2.04 (m, 1H), 1.29 (td, J=7.1, 2.6 Hz, 6H).

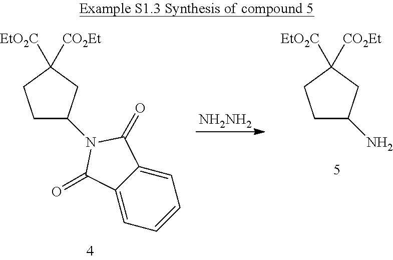 [SMILES: CCOC(=O)C1(C(=O)OCC)CCC(N2C(=O)c3ccccc3C2=O)C1 ; "NH2NH2" ; CCOC(=O)C1(C(=O)OCC)CCC(N)C1]

To a 5 L 3-necked flask equipped with a mechanic stirrer was added diethyl 3-(1,3-dioxoisoindolin-2-yl)cyclopentane-1,1-dicarboxylate (120 g, 334 mmol), MeOH (1580 mL), DCM (1050 mL), and hydrazine (121 mL, 1336 mmol) at room temperature. The reaction mixture was stirred at room temperature for 18 h. The reaction became a thick suspension, which was filtered. The filter cake was rinsed with DCM (500 mL), and the combined filtrates were concentrated in vacuo. To the residue was then added DCM (900 mL) and 1 N NaOH (440 mL). The organic phase was collected, washed with water (200 mL) and brine (200 mL), dried over Na₂SO₄, and filtered and concentrated in vacuo to give the product diethyl 3-aminocyclopentane-1,1-dicarboxylate as a slightly yellow oil (68 g). The combined water phase was back extracted with DCM to give additional 3 g product. The combined desired product was 71 g (93% yield), which was which was used directly for the next step without further purification.

LC-MS [M+1]: 230.

¹H NMR (400 MHz, CHLOROFORM-d) δ 4.19 (quin, J=7.1 Hz, 4H), 3.45 (t, J=6.7 Hz, 1H), 2.55-2.39 (m, 2H), 2.17 (dt, J=13.8, 7.9 Hz, 1H), 2.01-1.90 (m, 2H), 1.64-1.37 (m, 3H), 1.25 (td, J=7.1, 3.7 Hz, 6H).

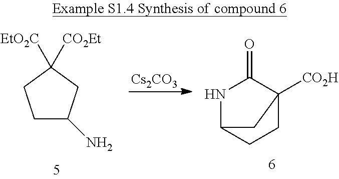

To a 2 L 3-neck flask with a condenser was added diethyl 3-aminocyclopentane-1,1-dicarboxylate (71 g, 310 mmol), cesium carbonate (202 g, 619 mmol) and MeOH (670 mL) under N₂. The reaction mixture was stirred at 65 °C for 4 h. The reaction was completed after 4 h and was cooled to room temperature and then concentrated in vacuo below 35 °C to remove MeOH to give a yellow oil. To the yellow residue was added H₂O (30 ml) and 2-MeTHF (120 mL), and the mixture was acidified with aqueous NaHSO₄ (149 g in 170 mL H₂O) to PH~3. Then the resulting solution was extracted with 2-MeTHF (200 mL×4). Combined extracts were concentrated by removal of solvent in vacuo to give a yellow solid. The solid was then suspended in benzene (500 mL) and heated with a Dean-Stark apparatus (Azeotropy) to remove water, which gave 43.5 g of yellow powder. The powder was co-rotovapped with toluene (200 mL×3) to remove any remaining moisture to offer the desired product (42.8 g, yield 89%) as a yellow powder, which was used without further purification for the next step reaction.

LC-MS [M+1]: 156.0.

¹H NMR (400 MHz, METHANOL-d4) δ 3.91-3.87 (m, 1H), 2.23-2.10 (m, 2H), 2.08-1.91 (m, 1H), 1.84-1.66 (m, 3H).

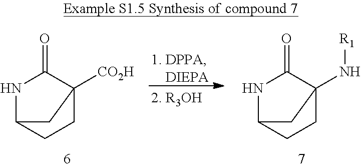

To a suspension of 3-oxo-2-azabicyclo[2.2.1]heptane-4-carboxylic acid (37.0 g, 238 mmol) in 2-Me-THF (610 mL) was added N,N-diisopropylethylamine (104 mL, 596 mmol) and diphenyl phosphorazidate (DPPA, 58.1 mL, 262 mmol). The reaction mixture was heated to 70 °C until it became a clear solution (within 5 min). LC/MS confirmed that the starting material was fully consumed within 20 min. After stirring at 70 °C for 50 min, benzyl alcohol (64.5 mL, 620 mmol) was added at 70 °C in one portion, and the reaction mixture was stirred at 80 °C for a total of 9 h until the reaction was completed. After cooling to room temperature, most of 2-MeTHF (~650 mL) was removed in vacuo. The residue was diluted with EtOAc (650 mL), washed with water (250 mL) and brine (200 mL), dried over Na₂SO₄, and filtered and concentrated in vacuo to give an extract as a yellow oil. The water and brine washes were combined and back-extracted with EtOAc (350 mL×8). The organic extracts were each dried, concentrated, and combined to give a crude product as a yellow oil (177 g). The crude was first purified on ISCO column chromatography with a 0-50% acetone/hexanes gradient followed by SFC to give two enantiomers:peak 1:23.7 g (yield 38.2%); and peak 2:23.5 g (yield 37.9%). LC/MS [M+1]: 260.7.

¹H NMR (400 MHz, CHLOROFORM-d) δ 7.39-7.31 (m, 5H), 5.69 (br s, 2H), 5.12 (br s, 2H), 3.87 (br s, 1H), 2.53-2.44 (m, 1H), 2.41 (br d, J=9.1 Hz, 1H), 2.06 (br s, 1H), 1.92 (dq, J=9.2, 2.3 Hz, 1H), 1.78-1.67 (m, 1H), 1.52-1.41 (m, 1H).

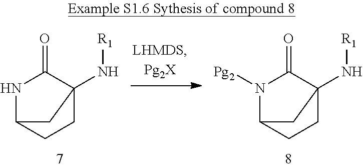

To a solution of benzyl (3-oxo-2-azabicyclo[2.2.1]heptan-4-yl)carbamate (1.66 g, 6.38 mmol) in THF (36 mL) was added LHMDS (1M in THF, 6.70 mL, 6.70 mmol) dropwise at 0~5 °C After stirring for 10 min, benzyl bromide (0.834 mL, 7.02 mmol) was added dropwise. The reaction was slowly warmed to room temperature and stirred for 20 h. Sat. NH₄Cl solution (20 mL) was added to quench the reaction. EtOAc (20 mL) was added next, and the organic layer was collected. The water layer was washed with EtOAc (15 mL). The organic layer and wash were combined and washed with brine (30 mL), dried over Na₂SO₄, filtered, and evaporated. The residue was purified by ISCO column chromatography with a 50-100% EtOAc/heptanes gradient to give benzyl(2-benzyl-3-oxo-2-azabicyclo[2.2.1]heptan-4-yl)carbamate (2.0 g, 5.71 mmol, 89% yield) as white solid.

LC/MS [M+1]: 351.3.

¹H NMR (400 MHz, CHLOROFORM-d) δ ppm 1.25-1.51 (m, 1H) 1.52-1.61 (m, 1H) 1.76-1.94 (m, 2H) 2.37 (br d, J=9.05 Hz, 1H) 2.47 (td, J=11.43, 4.28 Hz, 1H) 3.67 (br s, 1H) 4.10 (d, J=15.16 Hz, 1H) 4.73 (d, J=14.92 Hz, 1H) 5.13 (s, 2H) 5.79 (s, 1H) 7.23-7.41 (m, 10H)

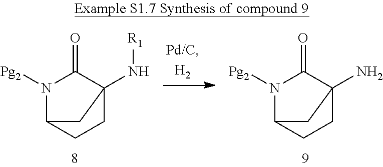

To a 250 mL three neck round bottom flask was charged a solution of benzyl(2-benzyl-3-oxo-2-azabicyclo[2.2.1]heptan-4-yl)carbamate (3.15 g, 8.99 mmol) in MeOH (30 mL). The solution was bubbled with N₂ for 10 min. 10% wet Pd/C (300 mg, 10% w/w) was added under N₂. The reaction mixture was then degassed until no bubbles was seen. A H₂ balloon was inserted, and the reaction mixture was stirred for 20 h. LC/MS indicated the completion of the reaction. The H₂ balloon was removed, and the reaction mixture was degassed until no bubbles seen. The flask was then filled with N₂. The mixture was filtered through a celite pad. The filtrate was concentrated in vacuo to give the product 4-amino-2-benzyl-2-azabicyclo[2.2.1]heptan-3-one (1.9 g, 8.78 mmol, 98% yield) as a colorless oil.

LC/MS [M+1]: 217.4.

¹H NMR (400 MHz, CHLOROFORM-d) δ ppm 1.55-1.63 (m, 2H) 1.66-1.92 (m, 6H) 3.58-3.61 (m, 1H) 4.03 (d, J=15.16 Hz, 1H) 4.73 (d, J=15.16 Hz, 1H) 7.25-7.37 (m, 5H).

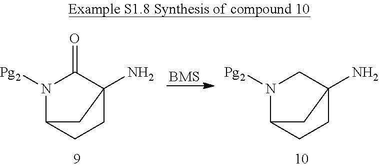

To a solution of 4-amino-2-benzyl-2-azabicyclo[2.2.1]heptan-3-one (320 mg, 1.480 mmol) in THF (15 mL) was added borane dimethyl sulfide complex (2.22 mL, 4.44 mmol) dropwise at room temperature. The reaction mixture was heated to 65 °C and stirred for 20 h. After cooling to room temperature, 6 N HCl solution (4.0 mL) was added to the reaction mixture to quench the reaction. The reaction mixture was then heated to 50 °C and stirred for 20 h. After cooling to room temperature, brine (20 mL) and EtOAc (20 mL) were added, followed by addition of 1 N NaOH to adjust the pH of the aqueous layer to ~14. DCM (20 mL×3) was then used to extract the amine product. The yield of 2-benzyl-2-azabicyclo[2.2.1]heptan-4-amine was assumed to be 100%.

LC/MS [M+1]: 203.4

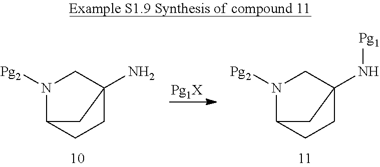

To a DCM solution (10 mL) containing ~300 mg of 2-benzyl-2-azabicyclo[2.2.1]heptan-4-amine was added di-tert-butyl dicarbonate (355 mg, 1.627 mmol) and stirred for 1 h. After removing DCM, the residue was purified by ISCO column chromatography with a 20-100% EtOAc/heptanes gradient to give tert-butyl (2-benzyl-2-azabicyclo[2.2.1]heptan-4-yl)carbamate (330 mg, 1.091 mmol, 73.8% yield).

LC/MS [M+1]: 303.3.

¹H NMR (400 MHz, CHLOROFORM-d) δ ppm 1.37-1.53 (m, 9H) 1.59-1.72 (m, 2H) 1.75-1.88 (m, 2H) 1.92-2.14 (m, 2H) 2.45-2.68 (m, 1H) 2.87-3.09 (m, 1H) 3.11-3.30 (m, 1H) 3.59-3.85 (m, 2H) 4.63-5.00 (m, 1H) 7.16-7.46 (m, 5H)

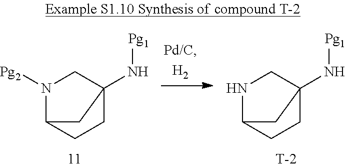

Tert-butyl (2-benzyl-2-azabicyclo[2.2.1]heptan-4-yl)carbamate (2.0 g, 6.61 mmol) was dissolved in ethanol (6 mL), to which was added 10% Pd(OH)₂/C (200 mg, 10% w/w). The reaction vessel was degassed, filled with N₂, degassed, and filled with H₂ with a H₂ balloon. The reaction mixture was then stirred for 18 h. The reaction vessel was then degassed and filled with N₂ multiple times. The reaction mixture was then filtered through a celite pad and concentrated in vacuo to give tert-butyl (2-azabicyclo[2.2.1]heptan-4-yl)carbamate (1.3 g, 6.12 mmol, 93% yield) as white solid.

LC/MS [M+1]: 213.3.

¹H NMR (400 MHz, CHLOROFORM-d) δ ppm 1.45 (s, 9H) 1.57-1.67 (m, 1H) 1.69-1.78 (m, 1H) 1.81-1.97 (m, 5H) 3.01 (s, 2H) 3.40 (s, 1H) 4.92 (s, 1H)

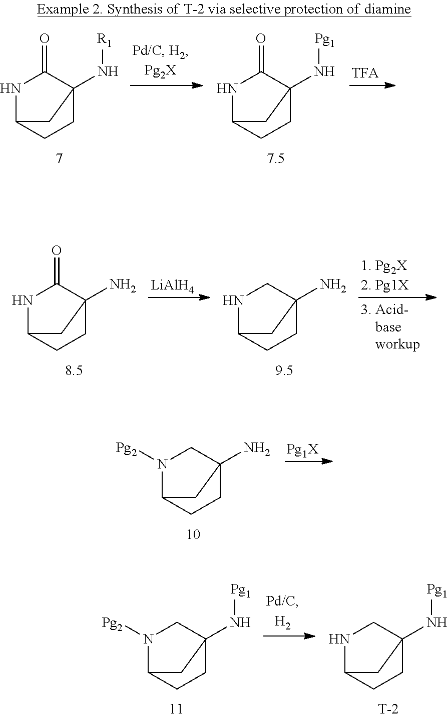

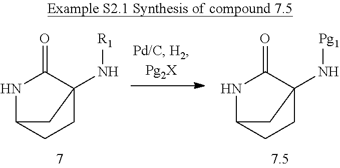

To a solution of benzyl (3-oxo-2-azabicyclo[2.2.1]heptan-4-yl)carbamate (5.2 g, 19.98 mmol) in MeOH (100 mL) was added di-tert-butyl dicarbonate (4.80 g, 21.98 mmol) and 10% Pd/C (0.52 g, 0.489 mmol, 10% W/W). The reaction vessel was degassed, filled with N₂, degassed, and filled with H₂ with a H₂ balloon. The reaction mixture was stirred for 18 h., and the reaction vessel was then degassed and filled with N₂ for multiple times. The reaction mixture was then filtered through a celite pad and concentrated in vacuo to give the crude product which was then triturated with 2-MeTHF at room temperature to −20 °C to give the product tert-butyl (3-oxo-2-azabicyclo[2.2.1]heptan-4-yl)carbamate (4.3 g, 19.00 mmol, 95% yield).

LC/MS [M+1]: 227.3

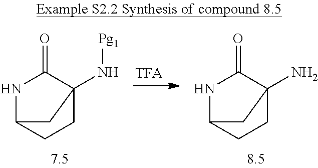

To a 100 mL flask was added tert-butyl (3-oxo-2-azabicyclo[2.2.1]heptan-4-yl)carbamate (872 mg, 3.85 mmol) and DCM (10 mL). The mixture was stirred at room temperature until all the solids were dissolved and then trifluoroacetic acid (2.97 mL, 38.5 mmol) was added. The mixture was stirred at room temperature overnight and concentrated in vacuo. The mixture was co-evaporated with toluene (10 mL) twice in vacuo to afford 4-amino-2-azabicyclo[2.2.1]heptan-3-one 2,2,2-trifluoroacetate (918 mg, 3.82 mmol, 99% yield).

¹H NMR (400 MHz, DMSO-d6) δ ppm 8.81 (br s, 3H) 8.36 (s, 1H) 3.86 (s, 1H) 1.98-2.09 (m, 1H) 1.84-1.96 (m, 2H) 1.76 (d, J=8.8 Hz, 1H) 1.52-1.68 (m, 2H)

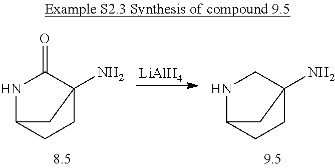

A 100 mL flask was charged with 4-amino-2-azabicyclo[2.2.1]heptan-3-one 2,2,2-trifluoroacetate (711 mg, 2.96 mmol) and THF (15 mL). The mixture was cooled in a water-ice bath, and lithium aluminum hydride (1M in THF) (14.80 mL, 14.80 mmol) was added dropwise. The mixture was stirred for 10 min and was allowed to reach room temperature by removing the bath. The mixture was heated at 55 °C (heating block temperature) for 5 h and stirred at room temperature overnight. The mixture was cooled to 0 °C and water (0.5 mL) was added dropwise. After stirring for 10 min, 15% NaOH solution (0.5 mL) was added followed by water (1.5 mL). The mixture was diluted with ether (15 mL) and stirred for 1 h. The suspension was filtered through a pad of celite and washed with ether (15 mL). The filtrate was dried with Na₂SO₄, filtered and concentrated to afford crude 2-azabicyclo[2.2.1]heptan-4-amine that was used without purification on the next step (assumed 100% yield).

¹H NMR (400 MHz, DMSO-d6) δ ppm 9.90 (br s, 1H) 9.36 (s, 1H) 9.16 (s, 2H) 8.96 (s, 1H) 3.96 (s, 1H) 3.09-3.28 (m, 2H) 1.83-2.11 (m, 6H) 1.71-1.83 (m, 1H)

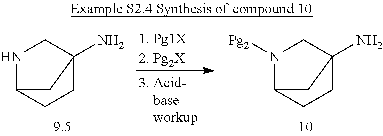

The crude 2-azabicyclo[2.2.1]heptan-4-amine (330 mg, 2.94 mmol) was dissolved in DCM (10 mL) and MeOH (10 mL), in which benzaldehyde (0.313 mL, 3.09 mmol) and magnesium sulfate (2.08 g, 17.28 mmol) were added. The mixture was stirred at room temperature overnight and then cooled down to 0 °C. A solution of Cbz-OSu (0.770 g, 3.09 mmol) in 1:1 MeOH/DCM (5 mL) was added dropwise. The mixture was stirred for 30 min at 0 °C, filtered through a pad of celite, and concentrated in vacuo. The residue was redissolved in 15 mL MeOH and 1N hydrochloric acid (15 mL, 15.00 mmol). The mixture was stirred for 30 min at room temperature to hydrolyze the imine. Most of methanol was then removed in vacuo and the aqueous layer was extracted with DCM (10 mL×2). The first extraction was discarded and the aqueous layer was basified with 1N NaOH solution to pH 12, followed by extraction with DCM (10 mL×3). The combined organic extracts were concentrated in vacuo to afford benzyl 4-amino-2-azabicyclo[2.2.1]heptane-2-carboxylate (367 mg, 1.490 mmol, 50.6% yield) as yellow oil.

¹H NMR (400 MHz, DMSO-d6) 7.27-7.52 (m, 5H) 5.04-5.25 (m, 2H) 4.09-4.35 (m, 1H) 3.09-3.28 (m, 2H) 1.35-1.98 (m, 8H)

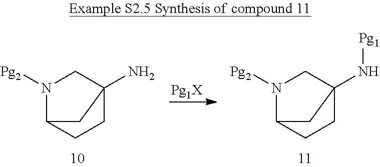

To a solution of benzyl 4-amino-2-azabicyclo[2.2.1]heptane-2-carboxylate (362 mg, 1.470 mmol) in DCM (10 mL) was added Boc-anhydride (385 mg, 1.764 mmol). The mixture was stirred at room temperature for 20 h. LCMS showed almost all of the SM was consumed. Solvent was evaporated and the residue was purified by ISCO column chromatography with a 20-100% EtOAc/heptanes gradient to afford benzyl 4-((tert-butoxycarbonyl)amino)-2-azabicyclo[2.2.1]heptane-2-carboxylate (478 mg, 1.380 mmol, 94% yield).

¹H NMR (400 MHz, DMSO-d6) 7.30-7.43 (m, 5H) 5.08-5.21 (m, 2H) 4.78-4.92 (m, 1H) 4.09-4.32 (m, 1H) 3.46-3.59 (m, 1H) 3.36-3.45 (m, 1H) 1.65-2.05 (m, 6H) 1.45 (s, 9H)

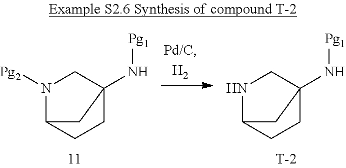

Benzyl 4-((tert-butoxycarbonyl)amino)-2-azabicyclo[2.2.1]heptane-2-carboxylate (478 mg, 1.380 mmol) in MeOH (10 mL) was hydrogenated overnight with a H₂ filled balloon in the presence of 10% Pd/C (50 mg, 0.047 mmol). The resulting mixture was filtered through a pad of celite, washed with methanol, and concentrated to afford tert-butyl (2-azabicyclo[2.2.1]heptan-4-yl)carbamate (284 mg, 1.338 mmol, 97% yield).

LC/MS [M+1]: 213.3.

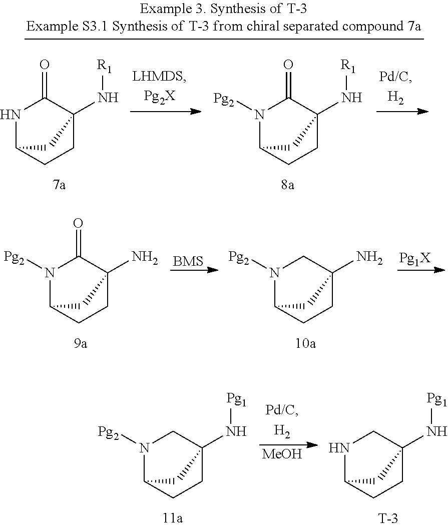

Compound T-3, an enantiomer of compound T-2, was prepared from an enantiomer 7a, obtained by chiral separation of compound 7. Each of the reaction procedures that led from compounds 7a to 8a, 8a to 9a, 9a to 10a, 10a to 11a, and 11a to T-3, were identical to those described in Examples 1.6, 1.7, 1.8, 1.9, and 1.10, respectively. In some embodiments, the kinetic resolution method used for chiral separation was Jacobsen's kinetic resolution. In some embodiments, the kinetic resolution method used for chiral separation was hydrolytic kinetic resolution catalyzed by chiral cobalt-salen complexes.

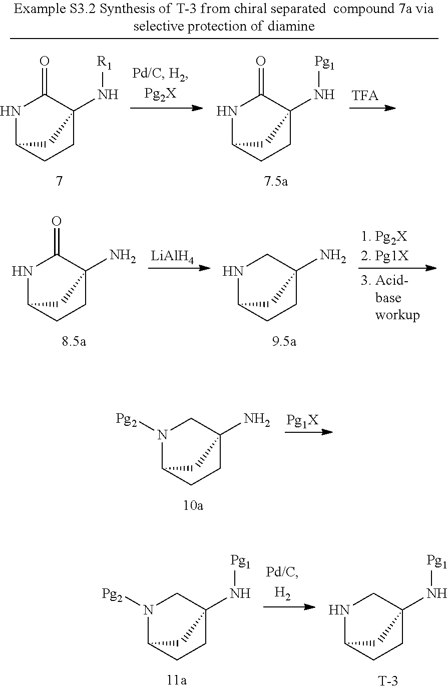

Compound T-3, an enantiomer of compound T-2, was prepared from an enantiomer 7a, obtained by chiral separation of compound 7. Each of the reaction procedures that led from compounds 7a to 7.5a, 7.5a to 8.5a, 8.5a to 9.5a, 9.5a to 10a, and 11a to T-3, were identical to those described in Examples 2.1, 2.2, 2.3, 2.4, 1.9, and 1.10, respectively. In some embodiments, the kinetic resolution method used for chiral separation was Jacobsen's kinetic resolution. In some embodiments, the kinetic resolution method used for chiral separation was hydrolytic kinetic resolution catalyzed by chiral cobalt-salen complexes.

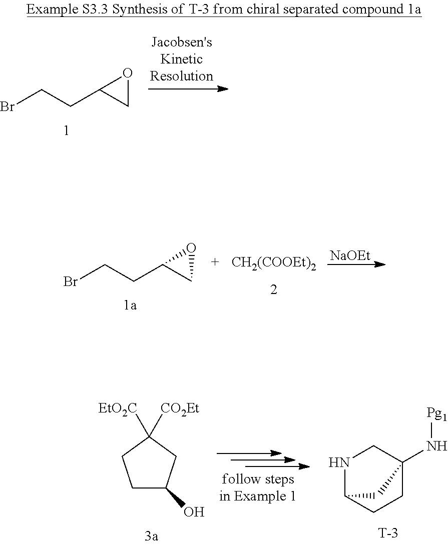

Compound T-3, an enantiomer of compound T-2, was prepared from an enantiomer 1a, obtained by chiral separation of compound 1 using Jacobsen's kinetic resolution. The reaction procedures were identical to those described in Example 1. The Jacobsen's kinetic resolution method used to prepare 1a is described below.

A 500 mL flask was charged with S,S—Co-Salen (6.12 g, 10.13 mmol) and DCM (60 mL). After stirring for 10 min at room temperature, nonafluoro-tert-butanol (7.08 mL, 50.7 mmol) was added dropwise, and the mixture was vigorously stirred open to air for 1 h. The mixture changed color from dark-red to black. The mixture was concentrated to dryness using a rotary evaporator. The solids were scraped from walls with spatula and dried in vacuo for 2 h to give the active catalyst. A separate 250 mL flask was charged with racemic 2-(2-bromoethyl) oxirane (102 g, 675 mmol), phenol (35.0 g, 372 mmol), and MTBE (50 mL). The mixture was cooled to 3-5 °C in an ice-water bath and then added to the flask containing the dry catalyst placed in a water/ice bath. The mixture was stirred in the bath for 30 min, then kept at 0~5 °C for 20 h. The flask was then allowed to warm to room temperature and MTBE was distilled off using a rotary evaporator (40 °C/70 mmHg). A short-path distillation adapter was then attached to the flask and the mixture was distilled at reduced pressure to give 47 g of the product (bp 66-68 °C/25 mmHg, then 43-45 °C/8 mmHg). At the end of distillation, the bath was heated to 100 °C and vacuum was lowered to 5-6 mmHg to maximize the recovery of the epoxide. NMR showed 89% purity. The crude material was re-distilled from a 100 mL flask using the same short-path distillation adapter to give(S)-2-(2-bromoethyl) oxirane (36.0 g, 212 mmol, 31.4% yield). Purity by NMR~95%.

¹H NMR (400 MHz, CHLOROFORM-d) δ=3.53 (dd, J=6.0, 7.4 Hz, 2H), 3.11 (dtd, J=2.7, 4.2, 6.8 Hz, 1H), 2.86 (dd, J=3.8, 4.9 Hz, 1H), 2.60 (dd, J=2.7, 4.9 Hz, 1H), 2.18 (dtd, J=4.6, 7.5, 15.0 Hz, 1H), 2.07 (qd, J=6.0, 15.0 Hz, 1H)

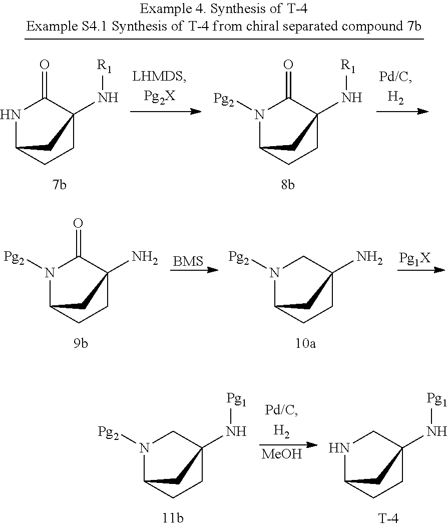

Compound T-4, an enantiomer of compound T-2, was prepared from an enantiomer 7b, obtained by chiral separation of compound 7. Each of the reaction procedures that led from compounds 7b to 8b, 8b to 9b, 9b to 10b, 10b to 11b, and 11b to T-4, were identical to those described in Examples 1.6, 1.7, 1.8, 1.9, and 1.10, respectively. In some embodiments, the kinetic resolution method used for chiral separation was Jacobsen's kinetic resolution. In some embodiments, the kinetic resolution method used for chiral separation was hydrolytic kinetic resolution catalyzed by chiral cobalt-salen complexes.

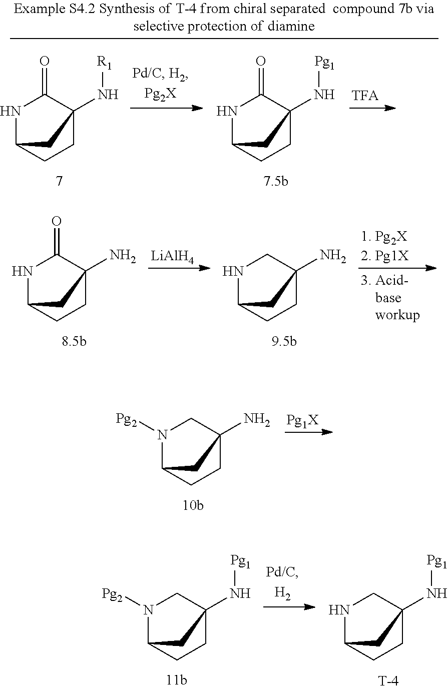

Compound T-4, an enantiomer of compound T-2, was prepared from an enantiomer 7b, obtained by chiral separation of compound 7. Each of the reaction procedures that led from compounds 7b to 7.5b, 7.5b to 8.5b, 8.5b to 9.5b, 9.5b to 10b, and 11b to T-4, were identical to those described in Examples 2.1, 2.2, 2.3, 2.4, 1.9, and 1.10, respectively. In some embodiments, the kinetic resolution method used for chiral separation was Jacobsen's kinetic resolution. In some embodiments, the kinetic resolution method used for chiral separation was hydrolytic kinetic resolution catalyzed by chiral cobalt-salen complexes.

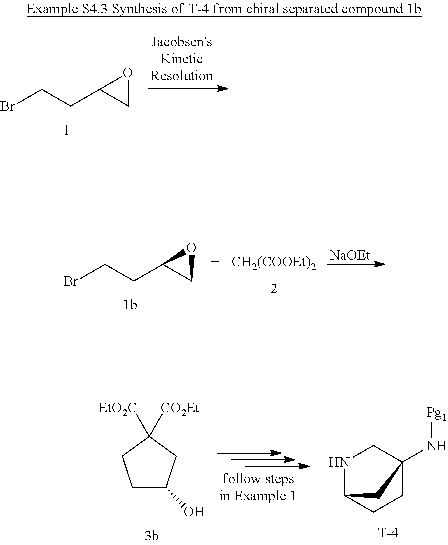

Compound T-4, an enantiomer of compound T-2, was prepared from an enantiomer 1b, obtained by chiral separation of compound 1 using Jacobsen's kinetic resolution. The reaction procedures were identical to those described in Example 1. The Jacobsen's kinetic resolution method was identical to that described in Example S3.3.

1. A method of preparing a compound of Formula (T-2): 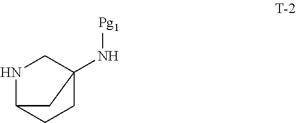
or a salt thereof, comprising:
reducing a compound of Formula (11): 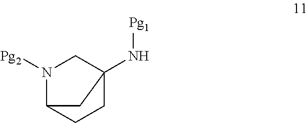
or a salt thereof,
wherein Pg₁ is a first protecting group; and
Pg₂ is a second protecting group;
to form the compound of Formula (T-2).

2. The method of claim 1, wherein:
a) the compound of Formula (T-2) is the compound of Formula (T-3): 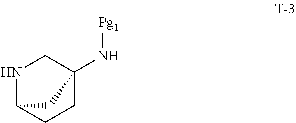
and the compound of Formula (11) is the compound of Formula (11a): 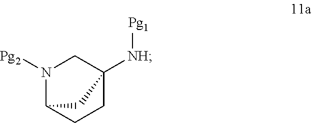
or
c) compound of Formula (T-2) is the compound of Formula (T-4): 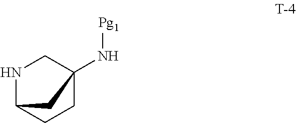
and the compound of Formula (11) is the compound of Formula (11b):

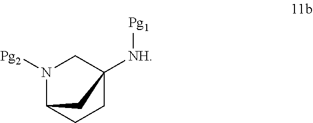

3. The method of claim 1 or claim 2, wherein the reduction of Formula (11), or a salt thereof, is catalyzed by a transition metal to form the compound of Formula (T-2), or a salt thereof.

4. The method of claim 3, wherein the transition metal is palladium chloride, palladium on barium sulfate, or palladium on carbon (Pd/C).

5. The method of any one of claims 1-4, wherein the reaction is carried out using an alcohol as solvent, optionally wherein the alcohol is methanol, ethanol, or isopropanol.

6. The method of any one of claims 1-5, wherein the compound of Formula (11), or a salt thereof, is prepared by protecting the primary amine group of a compound of Formula (10): 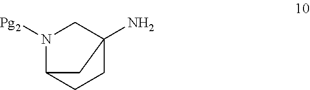
or a salt thereof.

7. A method of preparing a compound of Formula (11), 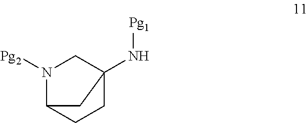
or a salt thereof, comprising:
protecting the primary amine group of a compound of Formula (10): 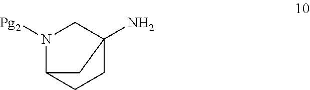
or a salt thereof, wherein:
wherein Pg₁ is a first protecting group; and
Pg₂ is a second protecting group;
to form the compound of Formula (11).

8. The method of any one of claims 1-7, wherein Pg₁ and Pg₂ are each independently selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.

9. The method of any one of claims 6-8, wherein the reaction is carried out using an organic solvent, optionally wherein the organic solvent is chloroform, dichloroethane, toluene, methanol, ethanol, isopropanol, or 2-methyl-THF, dioxane, ethyl acetate, or tetrahydrofuran.

10. The method of any one of claims 1-9, wherein the compound of Formula (10), or a salt thereof, is prepared by sequential protection of the substituent and heterocyclic amines of a compound of Formula (9.5): 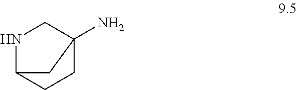
Or a salt thereof, with Pg₂ and Pg₁, respectively, followed by selective deprotection of the substituent amine to form the compound of Formula (10); or
wherein the compound of Formula (10), or a salt thereof, is prepared by reducing a compound of Formula (9): 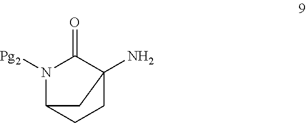
or a salt thereof, to form the compound of Formula (10).

11. A method of preparing a compound of Formula (10), 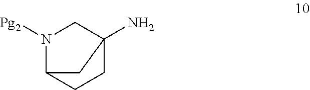
or a salt thereof, comprising:
reducing a compound of Formula (9): 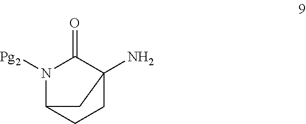
or a salt thereof,
wherein Pg₂ is a second protecting group, to form the compound of Formula (10), optionally wherein Pg₂ is selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.

12. The method of claim 10 or 11, wherein the reducing agent is a metal hydride, aluminum hydride, borohydride, or borane reagent, optionally wherein the reducing agent is lithium aluminum hydride, triphenylphosphine borane, borane tetrahydrofuran, or borane dimethylsulfide.

13. The method of any one of claims 10-12, wherein the reaction is carried out using an aprotic solvent, optionally wherein the aprotic solvent is diethyl ether, tetrahydrofuran, 2-methyltetrahydrofuran, acetonitrile, 1,4-dioxane, toluene, dichloromethane, dichloroethane, chloroform, or a mixture thereof.

14. The method of any one of claims 10-13, wherein the compound of Formula (9), or a salt thereof, is prepared by reducing a compound of Formula (8): 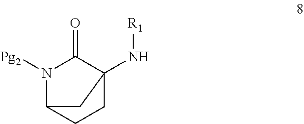
or a salt thereof, wherein:
R₁ is a benzyl carbamate optionally substituted by C₁-C₆ alkoxy, C₁-C₆ alkyl, halide, or an amino protecting group; and
Pg₂ is a second protecting group
to form the compound of Formula (9).

15. A method of preparing a compound of Formula (9), 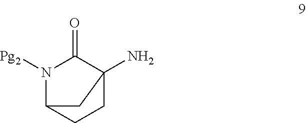
or a salt thereof, comprising:
reducing a compound of Formula (8): 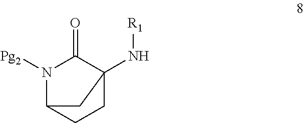
or a salt thereof, wherein:
R₁ is a benzyl carbamate optionally substituted by C₁-C₆ alkoxy, C₁-C₆ alkyl, or halide, or an amino protecting group; and
Pg₂ is a second protecting group
to form a compound of Formula (9), optionally wherein Pg₂ is selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.

16. The method of claim 14 or 15, wherein the reduction is catalyzed by a transition metal, optionally wherein the transition metal is palladium chloride, palladium on barium sulfate, or palladium on carbon (Pd/C).

17. The method of any one of claims 14-16, wherein the reaction is carried out using an alcohol as solvent, optionally wherein the alcohol is methanol, ethanol, or isopropanol.

18. The method of any one of claims 14-17, wherein the compound of Formula (8), or a salt thereof, is prepared by reacting a compound of Formula (7): 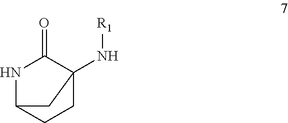
or a salt thereof, with Pg₂X, wherein:
R₁ is a benzyl carbamate optionally substituted by C₁-C₆ alkoxy, C₁-C₆ alkyl, halide, or an amino protecting group;
Pg₂ is a second protecting group; and
X is a leaving group;
to form the compound of Formula (8).

19. A method of preparing a compound of Formula (8), 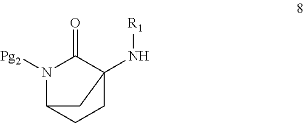
or a salt thereof, comprising:
reacting a compound of Formula (7): 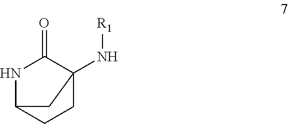
or a salt thereof, with Pg₂X, wherein:
R₁ is a benzyl carbamate optionally substituted by C₁-C₆ alkoxy, C₁-C₆ alkyl, halide, or an amino protecting group;
Pg₂ is a second protecting group; and
X is a leaving group;
to form the compound of Formula (8), optionally wherein Pg₂ is selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.

20. The method of claim 18 or 19, wherein the reaction is carried out in the presence of a non-nucleophilic base, optionally wherein the non-nucleophilic base is N,N-diisopropylethylamine, lithium diisopropylamide, sodium bis(trimethylsilyl)amide, sodium tert-butoxide, or lithium bis(trimethylsilyl)amide.

21. The method of any one of claims 18-20, wherein the reaction is carried out using an ether solvent, optionally wherein the ether solvent is tetrahydrofuran, 2-methyltetrahydrofuran, 1,4-dioxane, methyl tert-butyl ether, or a mixture thereof.

22. A method of preparing a compound of Formula (10), 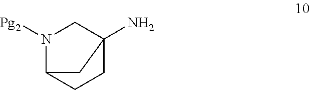
or a salt thereof, wherein Pg₂ is a second protecting group, comprising:
sequential protection of the substituent and heterocyclic amines of a compound of Formula (9.5): 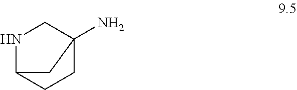
or a salt thereof, with Pg₂ and Pg₁, respectively, followed by selective deprotection of the substituent amine to form the compound of Formula (10), optionally wherein Pg₁ and Pg₂ are each independently selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.

23. The method of claim 10 or 22, wherein the protection reactions are carried out using an organic solvent, optionally wherein the organic solvent is chloroform, dichloromethane, tetrachloromethane, dichloroethane, toluene, methanol, ethanol, isopropanol, toluene, or tetrahydrofuran.

24. The method of any one of claims 10, 22, and 23, wherein the selective deprotection of the substituent amine is carried out with an acid, optionally wherein the acid is hydrochloric acid.

25. The method of any one of claim 10 or 22-24, wherein the compound of Formula (9.5), or a salt thereof, is prepared by reducing a compound of Formula (8.5), 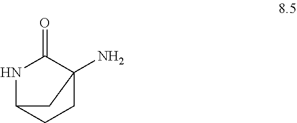
or a salt thereof, to form the compound of Formula (9.5).

26. A method of preparing a compound of Formula (9.5), 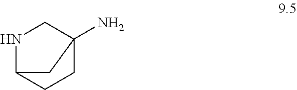
or a salt thereof, comprising:
reducing a compound of Formula (8.5): 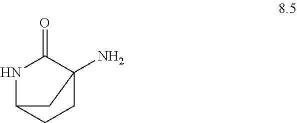
or a salt thereof, to form a compound of Formula (9.5).

27. The method of claim 25 or 26, wherein the reducing agent is a metal hydride, aluminum hydride, borohydride, or borane reagent, optionally wherein the reducing agent is lithium aluminum hydride, triphenylphosphine borane, borane tetrahydrofuran, or borane dimethylsulfide.

28. The method of any one of claims 25-27, wherein the reaction is carried out using an aprotic solvent, optionally wherein the aprotic solvent is diethyl ether, tetrahydrofuran, 2-methyltetrahydrofuran, acetonitrile, 1,4-dioxane, toluene, dichloromethane, dichloroethane, chloroform, or a mixture thereof.

29. The method of any one of claims 25-28, wherein the compound of Formula (8.5), or a salt thereof, is prepared by hydrolyzing a compound of Formula (7.5): 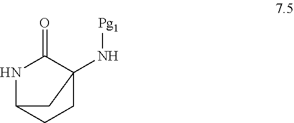
or a salt thereof, wherein Pg₁ is a first protecting group to form a compound of Formula (8.5).

30. A method of preparing a compound of Formula (8.5), 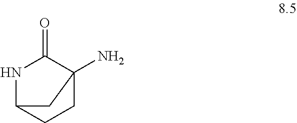
or a salt thereof, comprising:
hydrolyzing a compound of Formula (7.5): 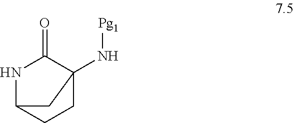
or a salt thereof, wherein Pg₁ is a first protecting group to form a compound of Formula (8.5), optionally wherein Pg₁ is selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.

31. The method of claim 29 or 30, wherein the hydrolysis is carried out with an acid, optionally wherein the acid is trifluoroacetic acid, hydrochloride, or phosphoric acid.

32. The method of any one of claims 29-31, wherein the protection reactions are carried out using an organic solvent, optionally wherein the organic solvent is chloroform, dichloromethane, tetrachloromethane, dichloroethane, toluene, methanol, ethanol, isopropanol, toluene, or tetrahydrofuran.

33. The method of any one of claims 29-32, wherein the compound of Formula (7.5), or a salt thereof, is prepared by deprotecting and subsequently protecting the primary amine group of a compound of Formula (7), 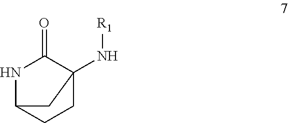
or a salt thereof.

34. A method of preparing a compound of Formula (7.5), 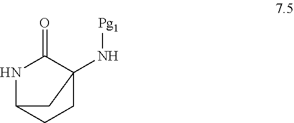
or a salt thereof, wherein Pg₁ is a first protecting group, wherein the method comprises:
deprotecting and subsequently protecting the primary amine group of a compound of Formula (7), 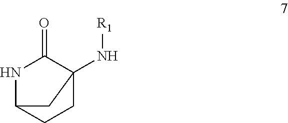
or a salt thereof, wherein R₁ is a benzyl carbamate optionally substituted by C₁-C₆ alkoxy, C₁-C₆ alkyl, halide, or an amino protecting group, to form the compound of Formula (7.5), optionally wherein Pg₁ is selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.

35. The method of claim 33 or 34, wherein the deprotection is catalyzed by a transition metal, optionally wherein the transition metal is palladium chloride, palladium on barium sulfate, or palladium on carbon (Pd/C).

36. The method of any one of claims 33-35, wherein the reaction is carried out using an alcohol as solvent, optionally wherein the alcohol is methanol, ethanol, or isopropanol.

37. The method of any one of claims 33-36, wherein the protection reaction is carried out using an organic solvent, optionally wherein the organic solvent is chloroform, dichloromethane, tetrachloromethane, dichloroethane, toluene, methanol, ethanol, isopropanol, toluene, or tetrahydrofuran.

38. The method of any one of claims 18, 19, and 33-37, wherein the compound of Formula (7), or a salt thereof, is prepared by reacting a compound of Formula (6): 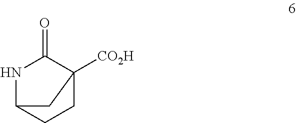 [SMILES: O=C(O)C12CCC(C1)NC2=O]
or a salt thereof,
with R₃OH, wherein R₃ is benzyl optionally substituted by C₁-C₆ alkoxy, C₁-C₆ alkyl, or halide, in the presence of diphenylphosphoryl azide and an amine base, to form the compound of Formula (7).

39. A method of preparing a compound of Formula (7), 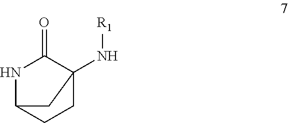
or a salt thereof, wherein R₁ is a benzyl carbamate optionally substituted by C₁-C₆ alkoxy, C₁-C₆ alkyl, halide, or an amino protecting group, comprising:
reacting a compound of Formula (6): 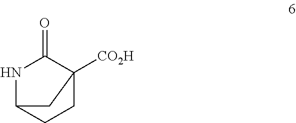 [SMILES: O=C(O)C12CCC(C1)NC2=O]
or a salt thereof, with R₃OH, wherein R₃ is a benzyl optionally substituted by C₁-C₆ alkoxy, C₁-C₆ alkyl, or halide, in the presence of diphenylphosphoryl azide and an amine base, to form the compound of Formula (7).

40. The method of claim 38 or 39, wherein the amine base is 2,2,6,6-tetramethylpiperidine, trimethylamine, triethylamine, or N,N-diisoproypylethylamine.

41. The method of any one of claims 38-40, wherein the reaction is carried out using an organic solvent, optionally wherein the organic solvent is dimethyl sulfoxide, dichloromethane, tetrahydrofuran, 2-methyltetrahydrofuran, or a mixture thereof.

42. The method of any one of claims 38-41, wherein the compound of Formula (7), or a salt thereof, is separated into enantiomers, a compound of Formula (7a) and a compound of Formula (7b), or salts thereof, using super fluid chromatography.

43. The method of any one of claims 38-42, wherein the compound of Formula (6), or a salt thereof, is prepared by reacting a compound of Formula (5): 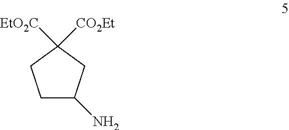 [SMILES: CCOC(=O)C1(C(=O)OCC)CCC(N)C1]
or a salt thereof, with a base to form the compound of Formula (6).

44. A method of preparing a compound of Formula (6), 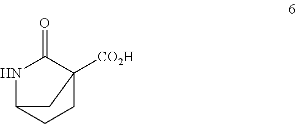 [SMILES: O=C(O)C12CCC(C1)NC2=O]
or a salt thereof, comprising:
reacting a compound of Formula (5): 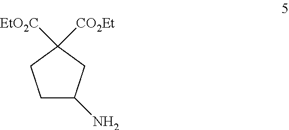 [SMILES: CCOC(=O)C1(C(=O)OCC)CCC(N)C1]
or a salt thereof, with a base to form the compound of Formula (6).

45. The method of claim 43 or 44, wherein the base is an inorganic base, optionally wherein the inorganic base is sodium tert-butoxide, potassium tert-butoxide, potassium carbonate, or cesium carbonate.

46. The method of any one of claims 43-45, wherein the reaction is carried out using an alcohol as solvent, optionally wherein the alcohol is methanol, ethanol, or isopropanol.

47. The method of any one of claims 43-46, wherein the compound of Formula (5), or a salt thereof, is prepared by deprotecting the amine of a compound of Formula (4): 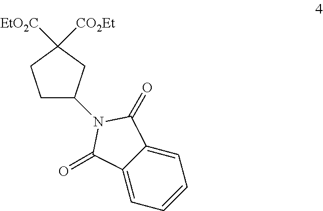 [SMILES: CCOC(=O)C1(C(=O)OCC)CCC(N2C(=O)c3ccccc3C2=O)C1]
or a salt thereof, to form the compound of Formula (5).

48. A method of preparing a compound of Formula (5), 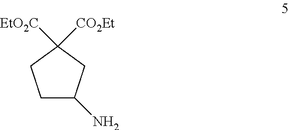 [SMILES: CCOC(=O)C1(C(=O)OCC)CCC(N)C1]
or a salt thereof, comprising:
deprotecting a compound of Formula (4): 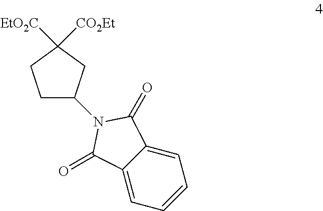 [SMILES: CCOC(=O)C1(C(=O)OCC)CCC(N2C(=O)c3ccccc3C2=O)C1]
or a salt thereof, to form the compound of Formula (5).

49. The method of claim 47 or 48, wherein the deprotection is carried out using a base, optionally wherein the base is sodium borohydride, methylamine, methylhydrazine, or hydrazine.

50. The method of any one of claims 47-49, wherein the reaction is carried out using a mixture of alcohol and an organic solvent, optionally wherein the alcohol is methanol, ethanol, or isopropanol, and the organic solvent is dimethyl sulfoxide, dichloromethane, tetrahydrofuran, or 2-methyltetrahydrofuran.

51. The method of any one of claims 47-50, wherein the compound of Formula (4), or a salt thereof, is prepared by reacting a compound of Formula (3): 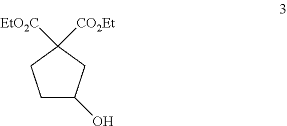 [SMILES: CCOC(=O)C1(C(=O)OCC)CCC(O)C1]
or a salt thereof, with phthalimide to form the compound of Formula (4).

52. A method of preparing a compound of Formula (4), 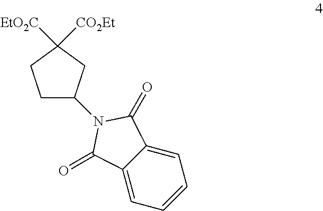 [SMILES: CCOC(=O)C1(C(=O)OCC)CCC(N2C(=O)c3ccccc3C2=O)C1]
or a salt thereof, comprising,
reacting a compound of Formula (3): 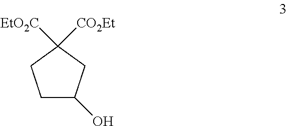 [SMILES: CCOC(=O)C1(C(=O)OCC)CCC(O)C1]
or a salt thereof, with phthalimide to form the compound of Formula (4).

53. The method of claim 51 or 52, wherein the reaction further comprises triphenylphosphine and diisopropyl azodicarboxylate.

54. The method of any one of claims 51-53, where in the reaction is carried out using an aprotic solvent, optionally wherein the aprotic solvent is diethyl ether, tetrahydrofuran, 2-methyltetrahydrofuran, acetonitrile, 1,4-dioxane, toluene, dichloromethane, dichloroethane, chloroform, or a mixture thereof.

55. The method of any one of claims 51-54, wherein the compound of Formula (3), or a salt thereof, is prepared by reacting a compound of Formula (1): 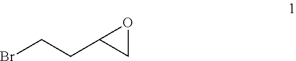
or a salt thereof,
and a compound of Formula (2): 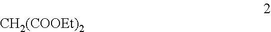
or a salt thereof, to form the compound of Formula (3).

56. A method of preparing a compound of Formula (3), 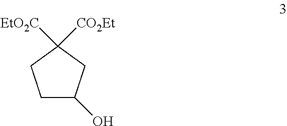 [SMILES: CCOC(=O)C1(C(=O)OCC)CCC(O)C1]
or a salt thereof, comprising:
reacting a compound of Formula (1): 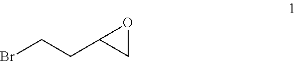
or a salt thereof,
and a compound of Formula (2): 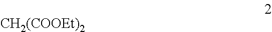
or a salt thereof, to form the compound of Formula (3).

57. The method of claim 55 or 56, wherein the reaction is carried out in the presence of a base, optionally wherein the base is sodium hydride, potassium tert-butoxide, or sodium ethoxide.

58. The method of any one of claims 55-57, wherein the reaction is carried out using an alcohol as solvent, optionally wherein the alcohol is methanol, ethanol, or isopropanol.

59. The method of any one of claims 55-58, wherein the compound of Formula (1), or a salt thereof, is separated into enantiomers, Formula (1a) and Formula (1b), or salts thereof, using kinetic resolution, optionally wherein the kinetic resolution is Jacobsen's kinetic resolution or hydrolytic kinetic resolution catalyzed by chiral cobalt-salen complexes.

60. A method of preparing a compound of Formula (T-2), or a salt thereof, comprising the following steps: 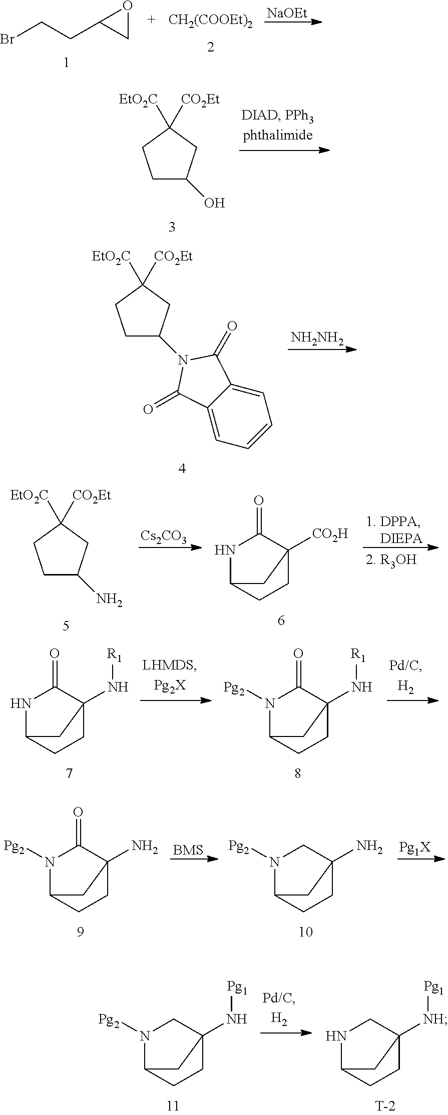
wherein:
R₃ is benzyl optionally substituted by C₁-C₆ alkoxy, C₁-C₆ alkyl, or halide;
R₁ is benzyl carbamate optionally substituted by C₁-C₆ alkoxy, C₁-C₆ alkyl, or halide;
Pg₁ is a first protecting group;
Pg₂ is a second protecting group; and
X is a leaving group.

61. A method of preparing a compound of Formula (T-2), or a salt thereof, comprising the following steps: 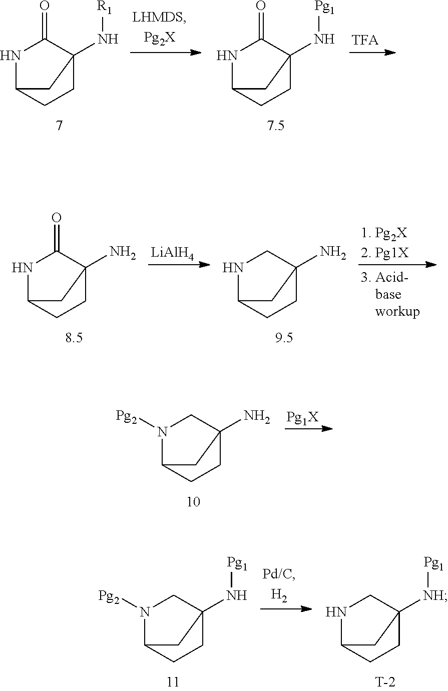
wherein:
R₁ is benzyl carbamate optionally substituted by C₁-C₆ alkoxy, C₁-C₆ alkyl, or halide;
Pg₁ is a first protecting group;
Pg₂ is a second protecting group; and
X is a leaving group.

62. The method of claim 156 or claim 157, wherein Pg₁ and Pg₂ are each independently selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.

63. A compound select from:
(a) a compound of Formula (T-3) or Formula (T-4): 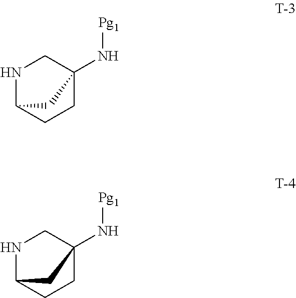
or a salt thereof;
(b) a compound of Formula (3): 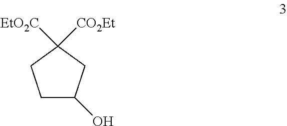 [SMILES: CCOC(=O)C1(C(=O)OCC)CCC(O)C1]
or a salt thereof, optionally wherein the compound of Formula (3), or a salt thereof, is a compound of Formula (3a): 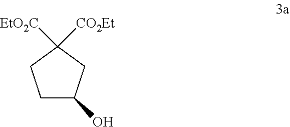 [SMILES: CCOC(=O)C1(C(=O)OCC)CC[C@H](O)C1]
or a salt thereof; or a compound of Formula (3b): 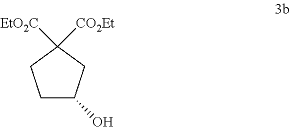 [SMILES: CCOC(=O)C1(C(=O)OCC)CC[C@@H](O)C1]
or a salt thereof;
(c) a compound of Formula (4): 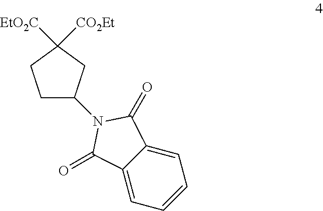 [SMILES: CCOC(=O)C1(C(=O)OCC)CCC(N2C(=O)c3ccccc3C2=O)C1]
or a salt thereof, optionally wherein the compound of Formula (4), or a salt thereof, is a compound of Formula (4a): 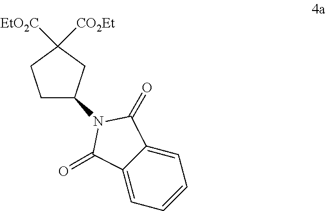 [SMILES: CCOC(=O)C1(C(=O)OCC)CC[C@H](N2C(=O)c3ccccc3C2=O)C1]
or a salt thereof; or a compound of Formula (4b): 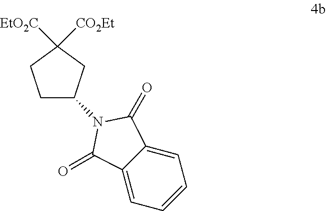 [SMILES: CCOC(=O)C1(C(=O)OCC)CC[C@@H](N2C(=O)c3ccccc3C2=O)C1]
or a salt thereof;
(d) a compound of Formula (5): 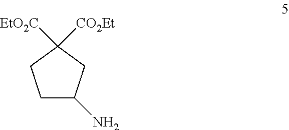 [SMILES: CCOC(=O)C1(C(=O)OCC)CCC(N)C1]
or a salt thereof, optionally wherein the compound of Formula (5), or a salt thereof, is a compound of Formula (5a): 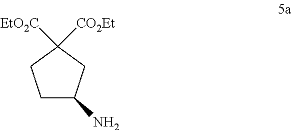 [SMILES: CCOC(=O)C1(C(=O)OCC)CC[C@H](N)C1]
or a salt thereof; or a compound of Formula (5b): 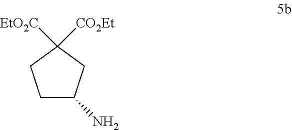 [SMILES: CCOC(=O)C1(C(=O)OCC)CC[C@@H](N)C1]
or a salt thereof;
(e) a compound of Formula (6): 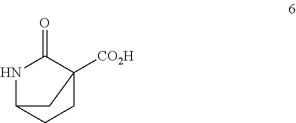 [SMILES: O=C(O)C12CCC(C1)NC2=O]
or a salt thereof, optionally wherein the compound of Formula (6), or a salt thereof, is a compound of Formula (6a): 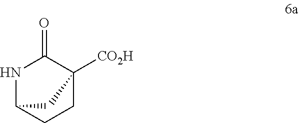 [SMILES: O=C(O)[C@]12CC[C@H](C1)NC2=O]
or a salt thereof; or a compound of Formula (6b): 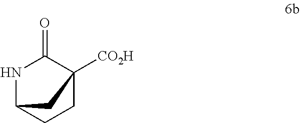 [SMILES: O=C(O)[C@@]12CC[C@@H](C1)NC2=O]
or a salt thereof.
(f) a compound of Formula (7): 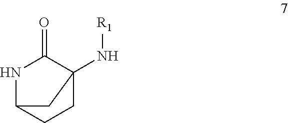
or a salt thereof, optionally wherein the compound of Formula (7), or a salt thereof, is a compound of Formula (7a): 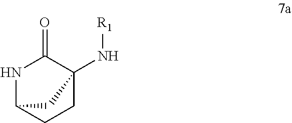
or a salt thereof; or a compound of Formula (7b): 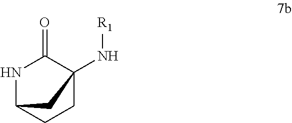
or a salt thereof;
(g) a compound of Formula (8): 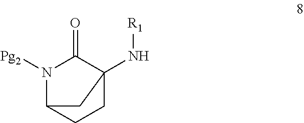
or a salt thereof, optionally wherein the compound of Formula (8), or a salt thereof, is a compound of Formula (8a): 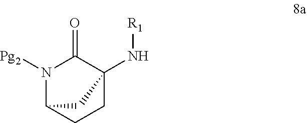
or a salt thereof; or a compound of Formula (8b): 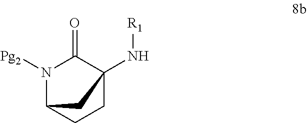
or a salt thereof.
(h) a compound of Formula (9): 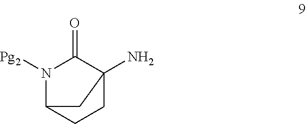
or a salt thereof, optionally wherein the compound of Formula (9), or a salt thereof, is a compound of Formula (9a): 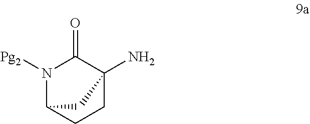
or a salt thereof; or a compound of Formula (9b): 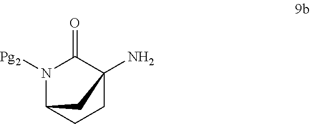
or a salt thereof.
(i) a compound of Formula (7.5): 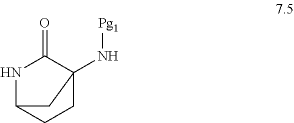
or a salt thereof, optionally wherein the compound of Formula (7.5), or a salt thereof, is a compound of Formula (7.5a): 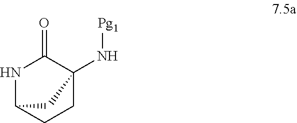
or a salt thereof; or a compound of Formula (7.5b): 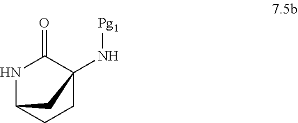
or a salt thereof;
(j) a compound of Formula (8.5): 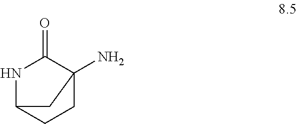
or a salt thereof, optionally wherein the compound of Formula (8.5), or a salt thereof, is a compound of Formula (8.5a): 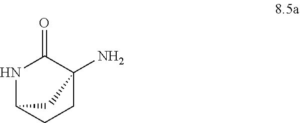
or a salt thereof; or a compound of Formula (8.5b): 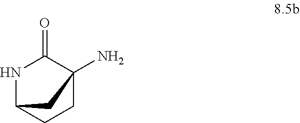
or a salt thereof;
(k) a compound of Formula (9.5): 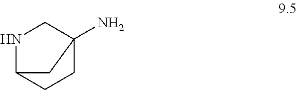
or a salt thereof, optionally wherein the compound of Formula (9.5), or a salt thereof, is a compound of Formula (9.5a): 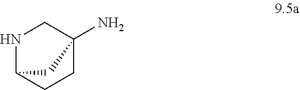
or a salt thereof; or a compound of Formula (9.5b): 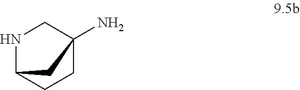
or a salt thereof;
(l) a compound of Formula (10): 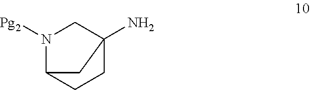
or a salt thereof, wherein Pg₂ is a second protecting group, optionally wherein the compound of Formula (10), or a salt thereof, is a compound of Formula (10a): 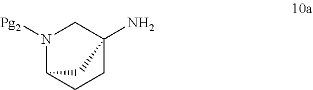
or a salt thereof; or a compound of Formula (10b): 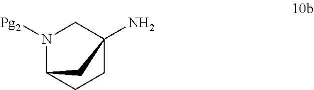
or a salt thereof; and
(m) a compound of Formula (11): 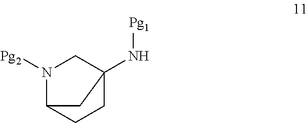
or a salt thereof, optionally wherein the compound of Formula (11), or a salt thereof, is a compound of Formula (11a): 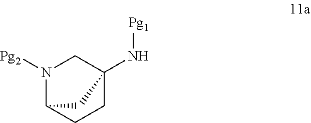
or a salt thereof; or a compound of Formula (11b): 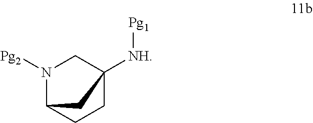
or a salt thereof;
wherein Pg₁ is a first protecting group; Pg₂ is a second protecting group; and R₁ is a benzyl carbamate optionally substituted by C₁-C₆ alkoxy, C₁-C₆ alkyl, halide, or an amino protecting group.

64. The compound of claim 63, wherein Pg₁ and Pg₂ are each independently selected from tert-butyloxycarbonyl, toluenesulfonyl, methyl, methoxymethyl, optionally substituted benzyl, optionally substituted carboxybenzyl, optionally substituted acyl, phthalimide, triphenylmethyl, diarylmethyl, benzylidene, and fluorenylmethyloxycarbonyl.
